(19) United States
(12) Patent Application Publication
(10) Pub. No.: US 2010/0233093 A1
(43) Pub. Date: Sep. 16, 2010

(54) Magnetic resonance imaging contrast agent with paramagnetic-inositol phosphates complexes

(75) Inventors: Byung Chul Oh, Incheon (KR); Hyeon Jin Kim, Incheon (KR)

Correspondence Address: CANTOR COLBURN, LLP, 20 Church Street, 22nd Floor, Hartford, CT, 06103, US

(73) Assignee: GACHON UNIVERSITY OF MEDICINE & SCIENCE INDUSTRY-ACADEMIC CORPERATION FOUNDATION, Incheon (KR)

(21) Appl. No.: 12/656,075

(22) Filed: Jan. 15, 2010

Related U.S. Application Data
(60) Provisional application No. 61/160,550, filed on Mar. 16, 2009.

(51) Int. Cl. A61K 49/10; C07F 9/06; A61P 35/00

(52) U.S. Cl. 424/9.32; 556/24

(57) ABSTRACT

Disclosed are novel MRI contrast agents and imaging methods using the same. The novel MRI contrast agent comprises a chelating molecule with at least two phosphate groups coordinated with at least one paramagnetic substances.

### TECHNICAL FIELD

The present invention relates, in general, to a novel magnetic resonance imaging contrast agent and, more particularly, to a novel magnetic resonance imaging contrast agent with at least two phosphate groups coordinated with at least one paramagnetic substance. Also, the present invention is concerned with an imaging method using the same.

### BACKGROUND ART

Phytate is the principal storage form of phosphorus in a great number of plant tissues, especially cereals such as rice, wheat, corn, beans, etc., accounting for 1-5% of the total weight of the cereal and for around 70-80% of the total phosphorous content of the cereal.

Phytate, a salt form of phytic acid, is a myo-inositol with up to six phosphates which bind mineral ions, such as Ca²⁺, Mg²⁺, Fe²⁺, and Zn²⁺, to form insoluble complexes which are generally bioavailable to individual animals, such as humans, chickens, swine, mice, etc., because they lack the digestive enzymes required to separate phosphorus from the insoluble complexes (Reddy et al 1982). Rather, undigested phytate acts as an anti-nutritional factor inhibiting the intestinal absorption of mineral ions such as Ca²⁺, Mg²⁺, Fe²⁺, and Zn²⁺. Therefore, the mineral-phytate complexes are known to give rise to mineral deficiencies in people whose diets rely on a high content of phytate food (Torre et al 1991).

Recent studies have shown that phytate purified from plant seeds has various beneficial effects including acting as an anticancer agent (Kennedy 1995; Kennedy & Manzone 1995) and antioxidant (Graf & Eaton 1990), and in relationship to the prevention of heart disease (Thompson 1994). Phytate may also participate in endochondral ossification impeding the mineralization of vesicles, a process believed to be regulated by enzymatic phytate hydrolysis (Caffrey et al 1999). Urinary phytic acid has an interesting beneficial effect on some pathological processes such as calcium urolithiasis by preventing, at very low concentrations, the development of renal calcifications. Phytate in a low concentration is reported to diminish the risk of calcium kidney stone recurrence (Grases et al 2000a) whereas a high amount of phytate increases the risk of developing urinary calcium stones (Grases et al 2000b). Recent research also has suggested that phytic acid is involved in the regulation of many important cellular functions as an activator of enzymes conducting DNA repair (Hanakahi et al 2000), as an activator of L-type Ca²⁺ channels (T J et al 1997), and as a regulator of mRNA export (York et al 1999).

In nuclear medicine, a colloid of phytic acid labeled with technetium-99 (99mTc) colloid, that is, Tc-phytate colloid, has extensively been used as a radionuclide imaging agent in positron emission tomography (PET) for the liver and spleen since 1973. The level and distribution of Tc-Phytate radiocolloid uptake in the liver, spleen, and bone marrow is a critical criteria for determining the progression and prognosis of chronic liver disease and monitoring hepatic function. For example, when administered with Tc-phytate, patients with chronic liver failure show significantly low levels of Tc-phytate in the liver, compared to healthy persons, this being presumably attributed to the functional failure of Kupffer cells (Hoefs et al 1995a; Hoefs et al 1997; Hoefs et al 1995b; Huet et al 1980).

In addition to liver function tests, Tc-phytate has also been utilized as a radiopharmaceutical for the mapping of sentinel lymph nodes (SLN) in patients with a variety of different types of cancer; breast cancer (Hino et al 2008; Ichihara et al 2003; Ikeda et al 2004; Kinoshita 2007; Koizumi et al 2006; Koizumi et al 2004a; Koizumi et al 2004b; Masiero et al 2005; Morota et al 2006; Noguchi 2001; Ohta et al 2004; Ohtake et al 2005; Takei et al 2006; Takei et al 2002; Tavares et al 2001; Tozaki et al 2003; Tsunoda et al 2002; Wada et al 2007; Yoshida et al 2002), malignant melanoma (Tavares et al 2001), vulvar cancer (Tavares et al 2001), squamous cell carcinoma (SCC) of the head and neck (Kosuda et al 2003; Ohno et al 2005), endometrial cancer (Nakayama et al 2004; Niikura et al 2004b; Niikura & Yaegashi 2004), cervical cancer (Nakayama et al 2004; Niikura et al 2004a; Silva et al 2005), pharyngeal carcinoma (Ohno et al 2005), prostate cancer (Nakayama et al 2004; Niikura et al 2004a; Silva et al 2005). The lymph nodes in which Tc-phytate is specifically absorbed and/or accumulated can be identified, allowing the examination of the metastasis of those lymph nodes proximal to a tumor region and thus cancer metastasis via biopsy. Among the advantages of the SLN identification technique, the first and foremost is to target metastasized lymph nodes only, with the minimal unnecessary dissection of benign lymph nodes, thereby reducing the risk of lymphedema, a common complication of this surgical procedure. In this regard, Tc-phytate in combination with PET imaging has been a method of choice for visualizing SLN in cancer patients. However, the need of repetitive exposure to PET in order to monitor patients with cancer is clearly a limitation of the method. In addition, PET can give information on cancer metastasis, but cannot point out the correct location of the metastasized lymph nodes due to its low resolution.

Magnetic resonance imaging (MRI) is a rapidly developing imaging modality wherein nuclear magnetic resonance (NMR) signals, mainly from water protons, are detected preceded by irradiating low electromagnetic energy into a sample. Thus far, the imaging modality of MRI has been regarded as being the most suitable for the diagnosis and monitoring of patients at multiple time points with short time intervals between imaging sessions because it requires a low amount of energy to excite water protons and thus MR images can be obtained non-invasively and with high spatial resolution (>10 times higher than PET). The intensity of the NMR signal is determined primarily by the amount of water protons. MRI contrast agents are often used to enhance image contrast.

Typically, MRI contrast agents are classified into paramagnetic and superparamagnetic agents according to their shortening of the T1 or T2 relaxation time of protons located nearby. Paramagnetic MRI contrast agents reduce T1 relaxation time, resulting in brighter images of tissue/organs while the reduction of T1 relaxation time by superparamagnetic MRI contrast agents results in darker images. Superparamagnetic iron oxide (SPIO) is typical of the MRI contrast agents altering T2 relaxation time (Low 1997). They are known to accumulate in organs because of macrophage uptake (Stoll et al 2004) and thus increase the contrast of images of the organs, but create dark images of the organs. Because a positive contrast (bright imaging) generally better defines the regions of interest and requires a shorter amount of time for MRI than does a negative contrast (dark imaging), the development of T1 agent will undoubtedly be of value.

Gadolinium (Gd) agents are representative of the T1 agents that are widely used in clinical practice. Most of the currently used gadolinium agents are in the form of chelates with ligands because gadolinium itself is highly toxic. For instance, Gd is chelated with diethylenetriamine pentaacetic acid (DTAP) to give a positive MRI contrast agent (Gd-DTAP). Additionally, the chelates of metals such as Mn²⁺ and Gd³⁺ have received special attention for their potential application as MRI T1 contrast agents. This effort has led to the development of extracellular MRI contrast agents that become distributed both in the vascular and extravascular space (Modo & BulteAime J. W. M. 2007).

Most of the T1 agents developed so far function to visualize the vessels of tissues, but are not specific to particular tissues or cells. There have been attempts made to develop tissue- or cell-specific MRI contrast agents by combining contrast agents with tissue- or cell-specific antibodies. For instance, tissue-specific MRI contrast agents were prepared by labeling monoclonal antibodies to the tissue with Gd-DTAP (Unger et al. 1985. Investigative Radiol 20(7):693-700). However, not only is the load level of Gd on the antibody very low (1.5 Gd³⁺/antibody molecule), but also no improvements of the image by antibody-DTPA-Gd were observed from models in vivo. As such, approaches conjugating targeting molecules such as antibodies directly with image contrast agents commonly suffer from the disadvantage of having to maintain the contrast agents at a low level; otherwise, the specificity of antibodies is negatively affected. The MRI contrast agent being delivered in a small amount results in low contrast of the desired image.

Conventionally, the retention time of image contrast agents is extended by the use of a polymer as a carrier of the contrast agent. For example, a carrier such as human albumin and polylysine is conjugated with DTPA which is then used to impregnate a metal of an MRI contrast agent therein (e.g., polylysine-bound Gd-DTAP, (Gerhard et al (1994), MRM 32:622-628)). Bovine serum albumin or bovine immunoglobuline is labeled with DTPA and Gd (Lauffer et al., (1985) Magnetic Resonance Imaging 3(1):11-16). A poly-L-lysine backbone-based polypeptide is reacted with DTPAa and the complex is used to chelate ¹¹¹In for MR imaging (Pimm et al (1992), Eur J Nucl Med 19:449-452). In these studies, the MRI contrast agents conjugated with the carriers were found to increase in retention time in blood pools. However, the conjugation of separate carriers to contrast agents is troublesome and requires additional expenses.

Therefore, there is a need for an MRI contrast agent that is tissue- or cell-specific, remains for an extended period of time in the body and retains thermodynamic and biological stability, and allows for clear T1 images (bright contrast). Leading to the present invention, intensive and thorough research into effective MRI contrast agents, conducted by the present inventors, resulted in the finding that a contrast agent in which a chelating molecule with at least two phosphate groups binds to a paramagnetic substance in a bi- or multidendate fashion is more stable and shows greater T1 contrast effect than do the currently used gadolinium (Gd) complexes in addition to being cells- or tissue-specific, so that it can be clinically applied to the diagnosis and clinical research of various diseases.

### DISCLOSURE

### Technical Problem

It is therefore an object of the present invention to provide an MRI contrast agent comprising a chelating molecule having at least two phosphate groups coordinated with at least one paramagnetic metal cation.

It is another object of the present invention to provide a method of visualizing an organ or tissue of a subject on a magnetic resonance image by administering the MRI contrast agent to the subject in need thereof and imaging the organ or tissue.

It is a further object of the present invention to provide a method for detecting macrophage activity at an organ or tissue of interest on a magnetic resonance image, comprising administering the MRI contrast agent to a subject in need thereof and imaging the organ or tissue, whereby the infection or inflammation of the organ or tissue can be diagnosed.

It is still a further object of the present invention to provide a method for non-invasively monitoring macrophage infiltration into sites of inflammation on a magnetic resonance image, comprising administering the MRI contrast agent to a subject in need thereof, and imaging the sites.

It is still another object of the present invention to provide a method for visualizing the presence of transplanted cells in vivo on a magnetic resonance image, comprising inserting the MRI contrast agent into cells; transplanting the cells to a site in a subject; and imaging the site using magnetic resonance imaging.

### Technical Solution

The present invention provides a cell-specific MRI contrast agent which is taken up by macrophages, has strong relaxivity and visualizing the tissue characteristics of a targeted region of interest. Also, the present invention provides an MRI contrast agent which can be easily administered, remain for a relative long period of time in the body (i.e. biodegradable with a reasonable half-life) and is free of cytotoxicity.

### ADVANTAGEOUS EFFECTS

The MRI contrast agents and the imaging methods using the same in accordance with the present invention enjoy the advantages of providing a readily injectable colloid suspension of inositol phosphate complexes containing paramagnetic or superparamagnetic metal ions, being organ-specific (e.g., the liver, spleen, lung and so forth), employing no detergents that may potentially cause allergic reactions, being thermodynamically stable compounds (e.g. more stable than EDTA and DTPA) which enable the contrast agent to be excreted intact, an important property since these contrast agents tend to be much less toxic than individual metal ions, and the ability to be injected in a relatively small dose (e.g. 1-4 μmol/kg) and still acquire good MR images because the inositol phosphate metal ion complexes of the invention concentrate in the targeted organ or tissues. Also, depending on the route of administration, the contrast agents of the present invention are distributed over different target tissues or organs and can be applied to the diagnosis of diseases specific for the organs or tissues. For example, when the contrast agents are administered intravascularly (e.g. intravenously or intra-arterially), they exert contrast effects on particular organs, including the liver, spleen, lymph nodes, bone marrow and lung, where the agents are taken up by macrophages. On the other hand, when the contrast agents are administered orally, they can enhance the contrast of the MR images of the gastrointestinal track. In addition, when the contrast agents are injected subcutaneously at the region of different types of cancer including breast, prostate and cervical cancer, they can be used to map metastatic sentinel lymph nodes since they specifically accumulate therein.

### DESCRIPTION OF DRAWINGS

The patent or application file contains at least one drawing executed in color. Copies of this patent or patent application publication with color drawings will be provided by the office upon request and payment of the necessary fee.

FIGS. 1A and 1B show Isothermal Titration Calorimetry (ITC) analysis of Gd³⁺ binding to phytate solution for analysis of the affinity, number and form of the binding. FIG. 1A is an illustration of Gd-phytate complexes in which Gd³⁺ ions bind to two oxianions from the phosphate groups of phytate in a bidentate fashion. FIG. 1B is ITC analysis of Gd³⁺ binding to phytate solution. The upper panel of FIG. 1B shows a calorimetric titration obtained when 10 mM Gd³⁺ is added intermittently in an amount of 1 μl each time into a 0.5 mM phytate solution (pH 7.0, 37° C.). The lower panel of FIG. 1B displays the heat exchanged per mole of titrant versus the ratio of total, concentration of the ligand to the total concentration of the phytate solution. The red solid line is the mathematically calculated best-fit for the two sets of models of the sites.

FIG. 2 shows ITC analysis of Me binding in the phytate solution for the affinity, number and form of the binding. The upper panel shows a calorimetric titration of 1.5 μl of 15 mM Mn²⁺ into a 0.5 mM phytate solution (pH 7.0, 37° C.). The lower panel displays the heat exchanged per mole of titrant versus the ratio of the total concentration of the ligand to the total concentration of the phytate solution. The red solid line is the mathematically calculated best-fit for the model of the sequential binding sites.

FIG. 3 shows ITC analysis of Ca²⁺ binding in the phytate solution for the affinity, number and form of the binding. The upper panel shows a calorimetric titration of 1.5 μl of 15 mM Ca²⁺ into a 0.5 mM phytate solution (pH 7.0, 37° C.). The lower panel displays the heat exchanged per mole of titrant versus the ratio of the total concentration of the ligand to the total concentration of the phytate solution. The red solid line is the mathematically calculated best-fit for the two sets of models of the sites.

FIG. 4 shows ITC analysis of Mg²⁺ binding in the phytate solution for affinity, number and form of the binding. The upper panel shows a calorimetric titration of 1.5 μl of 30 mM Mg²⁺ into a 0.5 mM phytate solution (pH 7.0, 37° C.). The lower panel displays the heat exchanged per mole of titrant versus the ratio of the total concentration of the ligand to the total concentration of the phytate solution. The red solid line is the mathematically calculated best-fit for the one set of models of the sites.

FIG. 5 shows ITC analysis of Gd³⁺ binding in the inositol-1,3,4-trisphosphate solution for affinity, number and form of the binding. The upper panel shows a calorimetric titration of 1.5 μl of 5 mM Gd³⁺ into 0.2 mM inositol-1,3,4-trisphosphate solution in 10 mM HEPES (pH 7.0, 25° C.). The lower panel displays the heat exchanged per mole of titrant versus the ratio of the total concentration of the ligand to the total concentration of the inositol-1,3,4-trisphosphate solution. The red solid line is the mathematically calculated best-fit for the one set of site models.

FIG. 6 shows ITC analysis of Gd³⁺ binding in the inositol-1,4,5-trisphosphate solution for affinity, number and form of the binding. The upper panel shows a calorimetric titration of 1.5 μl of 5 mM Gd³⁺ into 0.2 mM inositol-1,4,5-trisphosphate solution in 10 mM HEPES (pH 7.0, 25° C.). The lower panel displays the heat exchanged per mole of titrant versus the ratio of the total concentration of the ligand to the total concentration of the inositol-1,4,5-trisphosphate solution. The red solid line is the mathematically calculated best-fit for the one set of site model.

FIG. 7 shows ITC analysis of Gd³⁺ binding in the inositol-1,3,4,5-tetrakisphosphate solution for affinity, number and form of the binding. The upper panel shows a calorimetric titration of 1.5 μl of 5 mM Gd³⁺ into 0.2 mM inositol-1,3,4,5-tetrakisphosphate solution in 10 mM HEPES (pH 7.0, 25° C.). The lower panel displays the heat exchanged per mole of titrant versus the ratio of the total concentration of the ligand to the total concentration of the inositol-1,3,4,5-tetrakisphosphate solution. The red solid line is the mathematically calculated best-fit for the two sets of site models.

FIGS. 8A and 8B show. Isothermal Titration Calorimetry (ITC) analysis of Fe³⁺ binding to phytate solution for analysis of the affinity, number and form of the binding. FIG. 8A is ITC analysis of Fe³⁺ binding to phytate solution. The upper panel of FIG. 8A shows a calorimetric titration obtained when 5 mM Fe³⁺ is added intermittently in an amount of 1.5 μl each time into a 0.5 mM phytate solution (pH 7.0, 37° C.). The lower panel of FIG. 8A displays the heat exchanged per mole of titrant versus the ratio of total concentration of the ligand to the total concentration of the phytate solution. The red solid line is the mathematically calculated best-fit for the two sets of models of the sites. FIG. 8B is an illustration of Fe-phytate complexes in which Fe³⁺ ions bind to two oxianions from the phosphate groups of phytate in a bidentate fashion.

FIG. 9 shows the ITC analysis of Gd³⁺ binding to EDTA solution for affinity, number and form of the binding. The upper panel shows a calorimetric titration of 1.5 μl of 5 mM Gd³⁺ into 0.4 mM EDTA solution in 10 mM MES (pH 5.6, 25° C.). The lower panel displays the heat exchanged per mole of titrant versus the ratio of the total concentration of the ligand to the total concentration of the EDTA solution. The red solid line is the mathematically calculated best-fit for the one set of model sites.

FIG. 10 shows ITC analysis of Gd³⁺ binding to diethylene triamine pentaacetic acid (DTPA) solution for affinity, number and form of the binding. The upper panel shows a calorimetric titration of 1.5 μl of 5 mM Gd³⁺ into 0.2 mM DTPA solution in 10 mM sodium acetate (pH 4.8, 25° C.). The lower panel displays the heat exchanged per mole of titrant versus the ratio of the total concentration of the ligand to the total concentration of the DTPA solution. The red solid line is the mathematically calculated best-fit for the two sets of site models.

FIG. 11 shows ITC analysis of Gd³⁺ binding in the diethylene triamine pentaacetic acid (DTPA) solution for affinity, number and form of the binding. The upper panel shows a calorimetric titration of 1.5 μl of 5 mM Gd³⁺ into 0.2 mM DTPA solution in 10 mM Tris (pH 7.8, 25° C.). The lower panel displays the heat exchanged per mole of titrant versus the ratio of the total concentration of the ligand to the total concentration of the DTPA solution. The red solid line is the best-fit for the two sets of site models as calculated mathematically.

FIGS. 12A and 12B show the measurement of relaxation rates (R1) of Mn²⁺, Gd-DTPA, Mn-phytate and Gd-phytate complexes at different concentrations in a 9.4 T MRI scanner. (A) An example MR image of 24 phantoms of Mn²⁺, Gd-DTPA, Mn-phytate and Gd-phytate complexes are given at different concentrations with an inversion recovery time The patent or application file contains at least one drawing executed in color. Copies of this patent or patent application publication with color drawings will be provided by the office upon request and payment of the necessary fee. (T1) of 3000 ms. (B) The relaxation rates (R1) of those phantoms in sec are compared to one another. At all concentrations in this example, the relaxation rates (R1) of Mn-phytate and Gd-phytate are higher than those of Mn²⁺ and Gd-DTPA.

FIGS. 13A and 13B show MR signal changes with concentrations of Gd-phytate in the RAW 264.7 macrophage cell line at 9.4 T. (A) T1-weighted MR images of six phantoms containing macrophages are prepared using Gd-phytate solutions at concentrations of 0, 0.125, 0.25, 0.375, 0.5 and 0.75 mM. The signal intensity is clearly increased with the concentrations of Gd-phytate. (B) A schematic illustration shows in vivo the mechanism of targeting the region of interest.

FIGS. 14A to 14C show time-dependent changes in the MR signal of a rat liver after i.v. administration of Mn-phytate complex at 9.4 T. The T1-weighted MR images of the liver of a normal rat are shown (A) prior to, (B) 40 min after and (C) 1 day after the i.v. administration of Mn-phytate (5 μmol/kg). The signal intensity of the liver parenchyma tissue 40 min after the i.v. administration (B) was estimated to be increased by about 32% with respect to that of the liver prior to the administration (A). 24 Hrs after the administration (C), the signal intensity of the liver decreased down to the baseline, suggesting that the administered contrast agent was eliminated from the rat liver within 24 hrs.

FIG. 15 shows time-dependent changes in the MR signal of a rat liver after i.v. administration of Mn-phytate complex at 9.4 T. The T2-weighted MR images of the liver of a normal rat (A) prior to, (B) 3 hrs after, (C) 24 hrs after, and (D) 5 days after the i.v. administration of Gd-phytate (4 μmol/kg). The signal intensity of the liver parenchyma tissue 3 hrs (B) and 24 hrs (C) after the administration was estimated to be about 14% and 26% reduced with respect to that prior to the administration (A), respectively. 5 days after the administration, the reduced signal intensity of the liver increased up to ˜95% of the baseline, suggesting that the administered contrast agent was eliminated from the rat liver after approximately 5 days.

FIGS. 16A to 16D show time-dependent changes in the MR signal of a rat liver after i.v. administration of Gd-phytate complex at 1.5 and 4.7 T. The pre- and post-contrast T1-weighted MR images of the livers of normal rats at 1.5 T (A and B) and 4.7 T (C and D). A low dose of Gd-phytate (4 μmol/kg) was administered intravenously. The efficacy of Gd-phytate as a positive contrast agent is well demonstrated at these lower magnetic field strengths.

FIG. 17. shows macroscopic examinations of primary tumor and lymph nodes dissection. The size of primary tumor, implanted at the left forelimb, was about 1 cm. Left axillary lymph node and brachial lymph node were enlarged and contained a black spot. But right axillary and brachial lymph nodes remained unchanged.

FIG. 18. shows T1-weighted MR images of sentinel lymph node. The image of brachial lymph node pre-contrast (A), 4 hours (B), 8 hours (C), and 24 hours (D) after the subcutaneous injection of Fe-phytate. An arrow indicates the hypointense regions of sentinel lymph node.

FIG. 19. shows T2-weighted MR images of sentinel lymph node. The image of brachial lymph node pre-contrast (A), 4 hours (B), 8 hours (C), and 24 hours (D) after the subcutaneous injection of Fe-phytate. An arrow indicates the hypointense regions of sentinel lymph node. An arrow indicates the hypointense regions of sentinel lymph node.

FIG. 20. shows histopathological features of the left brachial lymph node obtained from the imaged mouse. (A) H&E stained for brachial lymph node, (B) Prussian blue stained for brachial lymph node, (C) the magnified view of the rectangular region of interest denoted in A. (D) the magnified view of the primary tumor. Scale bars, 500 μm (A, B); 100 μm (C, D).

### BEST MODE

In accordance with an aspect thereof, the present invention pertains to a magnetic resonance imaging contrast agent comprising a chelating molecule having at least two phosphate groups coordinated with at least one paramagnetic substances.

The term “magnetic resonance imaging (MRI)”, as used herein, refers to a hemodynamic-based medical imaging modality wherein nuclear magnetic resonance signals from water protons are detected after irradiating low electromagnetic energy to a sample. It is based on molecular science in terms of utilizing magnetic resonance signals as well as on hemodynamics and differs from positron emission tomography. (PET) which is based on radionuclides.

The term “contrast agent” refers to a medium used to enhance the contrast of internal body structures such as vessels or organs to thereby improve the visibility thereof when performing magnetic resonance imaging. A contrast agent is helpful in qualitatively and quantitatively determining disease and/or injury by improving the visibility and contrast of the surface of an object of interest.

As used herein, the term “paramagnetic substance” means a substance which forms a magnetic moment upon the application of an external magnetic field thereto whereas magnetization is not retained in the absence of an externally applied magnetic field because thermal motion causes the spin of unpaired electrons to become randomly oriented without it. By taking advantage of its property of shortening the magnetic relaxation time of water molecules, a paramagnetic substance is usable as an active component of MRI contrast agents.

Preferably, the paramagnetic substance useful in the present invention is a transition element. More preferably, it is selected from a group consisting of Cr³⁺, Co²⁺, Mn²⁺, Ni²⁺, Fe²⁺, Fe³⁺, Cu²⁺ and Cu³⁺, with the strongest preference being given for Fe²⁺, Fe³⁺ and Mn²⁺.

Alternatively, the paramagnetic element is a lanthanide element. Preferably, the lanthanide element is selected from a group consisting of La³⁺, Gd³⁺, Ce³⁺, Tb³⁺, Pr³⁺, Dy³⁺, Nd³⁺, Ho³⁺, Pm³⁺, Er³⁺, Sm³⁺, Eu³⁺, Yb³⁺ and Lu³⁺ with the strongest preference being for Gd³⁺.

Instead of the paramagnetic substances, isotopes, whether radioactive or paramagnetic, may be used as well. Examples of the radioactive isotopes useful in the present invention include, but are not limited to, ¹¹C, ¹³N, ¹⁸F, ¹²³I, ¹²⁴I, ¹²⁵I, ^{99m}Tc, ¹¹¹In, ⁷⁶Br, ⁶²Cu, ⁶⁷Ga and ⁶⁸Ga.

In accordance with the present invention, the contrast agent causes a reduction in the T1 or T2 relaxation time near the region of interest within the body of the subject.

As used herein, the terms “chelating molecule” or “chelator” refer to a compound or chemical residue capable of binding to a metal ion via at least one donor atom. The ability of a paramagnetic chelate complex to reduce the T1 magnetic relaxation time and stabilize the paramagnetic substance depends utterly on the chemical structure of the chelating molecule coordinating with the paramagnetic substance.

In this context, the chelating molecule useful in the present invention has at least two phosphate groups as preferably exemplified by phytate, inositol phosphate and phosphatidylinositol phosphate. More preferable examples of the chelating molecule include d-myo-inositol-1,2,3,4,5,6-hexakisphosphate, d-myo-inositol-1,2,3,4,5-pentaphosphate, d-myo-inositol-1,3,4,5-tetraphosphate, d-myo-inositol-1,4,5-trisphosphate, d-myo-inositol-1,3,4-trisphosphate, phosphatidylinositol-3,4,5-trisphosphate and phosphatidylinositol-4,5-bisphosphate.

To be suitably used under physiological conditions, the chelating molecule has a pH ranging from 4 to 9 and most suitably from 6 to 8.

In one embodiment of the present invention, the molar ratio of the metal to the chelating molecule for the MRI contrast agent ranges from 0.5 to 3, or a molar ratio of the chelating molecule to the metal ranges from 0.5 to 3.

When the contrast agent of the present invention is used in diagnostic conditions, it is not desirable that the chelating molecule bind to calcium endogenously present in the blood vessel. Therefore, to minimize the chelation of blood Ca²⁺ by the chelating molecule of the contrast agent when it is used diagnostically in a subject, Ca²⁺ ions may be included in the contrast agent upon the preparation thereof. So long as it sufficiently prevents the contrast agent from harmfully chelating away endogenous calcium in the body of the subject during a diagnostic MRI or PET imaging session, any amount of calcium may be mixed with the contrast agent. A amount of Ca²⁺ preferred is in an equimolar concentration with the contrast agent.

The contrast agent of the present invention may be prepared from the paramagnetic substance and the chelating molecule using a method well known in the art. For example, a chelating molecule with at least two phosphate groups is dissolved in sterile water, adjusted to the desired pH value, and mixed with a paramagnetic ion colloid.

In another method, a paramagnetic phytate complex solution is dried at a low temperature together with the calcium ion and stored in a container. A paramagnetic phytate complex solution and a calcium ion solution may be separately dried at low temperature and stored in a single container or in respective separate containers before being used together. Alternatively, a phytate solution is first placed in a container, followed by the addition of paramagnetic ion thereto. The paramagnetic phytate complex may be in powder as well as solution form. In the contrast agent of the present invention, the paramagnetic phytate chelate may optionally be conjugated with a monoclonal antibody specific for certain tumor.

In accordance with another aspect thereof, the present invention pertains to a method of visualizing an organ or tissue of a subject in need thereof on a magnetic resonance image, comprising administering the MRI contrast agent to the subject and imaging the organ or tissue.

As used herein, the terms “administration” or “administering” are intended to mean the action of introducing a desired material into a subject via a suitable route. The effective amount and administration route are dependent on various factors including the patient's age, body weight and on the site to be treated, as well as the kind of contrast agent to be used, diagnostic uses to be considered, and formulation forms (e.g., suspensions, emulsions, microspheres, liposomes, etc.) of the contrast agent, which is already apparent to those skilled in the art. Typically, the amount of a contrast agent administered is low at first, and is gradually increased until a desired diagnostic result is obtained. Imaging may be achieved using a typical technique known to those skilled in the art. Generally, an aqueous solution of a contrast agent is intravenously administered to a subject at a dose of from about 10 to 1,000 μmole of the contrast agent per kg weight (within the range, all dose combinations, sub-combinations, and particular doses are possible).

The contrast agent composed of Tc-phytate and paramagnetic cation in accordance with an embodiment of the present invention may be applied to positron emission tomography (PET) imaging as well as magnetic resonance imaging (MRI).

In the visualizing method, the MRI contrast agent is preferably administered intravenously when the subject is suspected of being affected, with fatty liver, liver cirrhosis or atherosclerosis.

In order to determine the metastasis of a tumor via the image of a sentinel lymph node, the MRI contrast agent may be administered subcutaneously.

Also, the MRI contrast agent may be administered when the organ or tissue is suspected of being a tumor. Preferably, the tumor may be breast cancer, malignant melanoma, vulvar cancer, squamous cell carcinoma (SCC) of the head and neck, endometrial cancer, cervical cancer, pharyngeal carcinoma, liver cancer, gastric cancer, colorectal cancer or prostate cancer.

Also, the MRI contrast, for example Fe-phytate complex, with anticancer drugs like doxorubicin (trade name Adriamycin; also known as hydroxydaunorubicin) for the treatment of various tumor, wherein doxorubicin Fe-phytate complex can subcutaneously administrate in the primary tumor of a group consisting of breast cancer, malignant melanoma, vulvar cancer, squamous cell carcinoma (SCC) of the head and neck, endometrial cancer, cervical cancer, pharyngeal carcinoma, liver cancer, gastric cancer, large intestine cancer, or prostate cancer.

Compared to conventional ones, the MRI contrast agent of the present invention has the advantages of being stable both thermodynamically and biologically, being tissue- or cell-specific, providing for clear T1 contrast effects (bright contrast) with high relaxivity, and showing a long retention time within the body.

First, thanks to the strong binding affinity of the chelating molecule for the paramagnetic substance, the contrast agent of the present invention is more stable than are conventional ones. In the following example, ITC thermodynamic assays were performed to measure the binding affinity of phytate or inositol phosphate for paramagnetic ions, indicating that phytate or inositol phosphate strongly binds with Gd ions in a bidendate mode and also with manganese ions and that inositol phosphate derivatives strongly bind Gd ions as well. Thus, phytate complexes with paramagnetic substance are very stable and suitable for use as contrast agents (see Tables 1 to 4).

The binding affinities of Gd³⁺ for phytate are about 10-fold higher than that of Gd³⁺ for DTPA and 10- to 100-fold higher than that of Gd³⁺ for EDTA, suggesting that Gd-phytate complexes according to the present invention are more stable and kinetically inert than both the conventional contrast agents Gd-EDTA and Gd-DTPA (see Table 5, below).

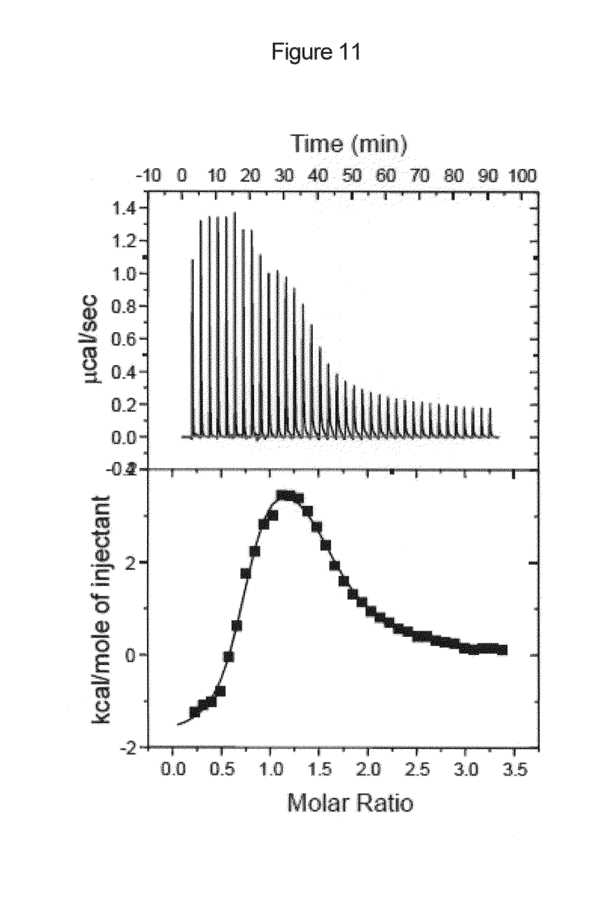

Second, the contrast, agent of the present invention efficiently enhances image contrast at a local site in MRI. In the following examples, the relaxation rates (R1=1/T1) of Mn-phytate and Gd-phytate were estimated at different concentrations and compared to those of Mn²⁺ and Gd-DTPA which are widely utilized in MR research. Mn-phytate and Gd-phytate were found to more effectively perturb local magnetic fields than do Mn²⁺ and Gd-DTPA, respectively, at almost all of the concentrations tested (see FIG. 11). As such, paramagnetic contrast agents with high T1 relaxivity show contrast effects even at relatively low doses which are the same as those of the conventional contrast agents, and this results in a significant reduction in the amount of the heavy metal Gd that is usually used as a paramagnetic substance, thus curtailing the potential danger of injuring the body. Guaranteeing contrast enhancement even with a small amount, the contrast agents according to the present invention are of great significance in MRI search.

Third, the contrast agents of the present invention stay for a longer period of time in the body and are much more quickly removed from the body. In the following examples, the changes in the signal intensity of the liver were observed to reach a maximum (up to ˜32% enhancement) about 40 minutes after the administration of Mn-phytate. The agent of the present invention then appeared to be quickly removed from the liver in about 24 hrs which is significantly shorter than other intracellular agents reported to date (e.g., SPIO). Accordingly, the contrast agents of the present invention are more efficient than the conventional contrast agents which need separate carriers in order to increase retention time.

Finally, the contrast agents of the present invention allow for tissue- or cell-specific imaging. The phagocytosis of Gd-phytate complexes of the present invention by macrophages, which is proven in the following example, can be utilized to visualize tissues or organs of interest upon the diagnosis of particular diseases.

Macrophages are found in abundance in the lymph nodes and the spleen. Macrophages, such as the Kupffer cells of the liver and the histiocytes of muscle tissues, are responsible for the uptake of contrast agents. Concerning the contrast agents of the present invention, they are taken up by Kupffer cells from blood vessels and perturb a local magnetic field to produce image contrast. Introduced into specific tissues through phagocytosis by macrophages, the contrast agents of the present invention can be therefore applied to the diagnosis of various diseases such as atherosclerotic plaques, organ graft rejection, multiple sclerosis, and the sentinel lymph node detection of various types of cancer.

In accordance with a further aspect thereof, the present invention pertains to a method for detecting macrophage activity in an organ or tissue of interest on a magnetic resonance image by administering the MRI contrast agent to a subject in need thereof and imaging the organ or tissue, whereby infection or inflammation of the organ or tissue can be diagnosed.

In accordance with still a further aspect thereof, the present invention pertains to a method for noninvasively monitoring macrophage infiltration into sites of inflammation on a magnetic resonance image by administering the MRI contrast agent to a subject in need thereof, and imaging the sites.

In accordance with still another aspect thereof, the present invention pertains to providing a method for visualizing the presence of transplanted cells in vivo on a magnetic resonance image by inserting the MRI contrast agent into cells, transplanting the cells to a site in a subject, and imaging the site using magnetic resonance imaging.

### MODE FOR INVENTION

A better understanding of the present invention may be obtained through the following examples which are set forth to illustrate, but are not to be construed as limiting the present invention.

### Example 1

### Binding Thermodynamics of Paramagnetic Ions to Phytate or Inositol Phosphate

ITC experiments were performed in a Microcal 200 isothermal titration microcalorimeter (Microcal, Inc., Northhampton, Mass. USA) to quantify the binding isotherms of paramagnetic metal ions such as Gd³⁺, Mn²⁺ and Ca²⁺ to phytate solution or inositol phosphate derivatives. Data collection, analysis and plotting were performed with resort to the software package Origin, version 7.0, supplied by Microcal. Titration in the microcalorimeter is based on the differential heat between a sample cell and a reference cell for each time a paramagnetic substance is injected thereinto. The reference cell is filled with distilled water. In a typical titration method, 1˜5 μl aliquots of a 5˜10 mM paramagnetic metal ion solution are injected at regular intervals of 2 min into phytate solutions each of a different pH while measuring differences in binding energy. The syringe was stirred at 1,000 rpm to form a stable bond between the phytate sample and the paramagnetic substance. The heat absorbed or released at each injection was measured by the calorimeter. These titration isotherms were integrated to provide the enthalpy change upon each injection. Isotherms were analyzed to fit well to two mathematically different sets of site models. Through the calorimetric analysis, parameters including the binding constant (Kₐ), the change in enthalpy (ΔH), and the stoichiometry of binding (N) were calculated. The change in free energy (ΔG) and change in entropy (LS) were then determined using the equation: ΔG=−RTln Kₐ=ΔH−TΔS, wherein R is the universal gas constant and T is the temperature in degrees Kelvin.

### Example 1.1

### ITC Analysis of Phytate with Gadolinium (Gd³⁺)

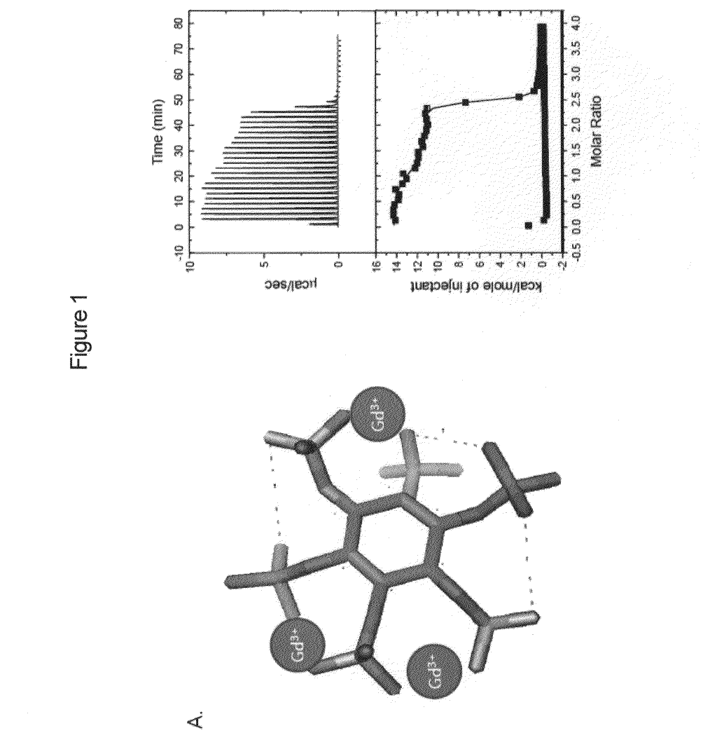

Calorimetric titration of phytate (0.5 mM) with a Gd³⁺ solution (10 mM) was measured at 37° C. as a function of pH ranging from 3.0 to 8.0. The binding isotherm, which corresponds to a plot of integrated heats as a function of the molar ratio of Gd³⁺/Phytate, is represented in FIG. 1B. In this figure, the solid line corresponds to the best-fit curve of the data between the phytate and gadolinium ion into the two different sets of site models, as mathematically determined, indicating ligand (Gd³⁺) binding to three non-identical independent sites. From analysis of the data, the dissociation constant (K_{d}), the number of Gd³⁺ bound per phytate molecule (n), and the association enthalpy change (ΔH) were obtained. The thermodynamic parameters corresponding to Gd³⁺ binding to phytate solution are given in Table 1. As is apparent from the data of Table 1, Gd³⁺ ions strongly bind two oxianions from the phosphate groups of phytate in a bidentate fashion (FIG. 1A) to form Gd-phytate complexes with high affinity of 10⁻⁹-10⁻⁷ M.

TABLE 1

| Parameters | Site | pH 3 | pH 4 | pH 5 | pH 6 | pH 7 | pH 8 |
|---|---|---|---|---|---|---|---|
| K_{d1} (10⁻⁹ M) | I | 73.0 | 8.9 | 12.8 | 26.3 | 12.9 | 7.8 |
| n₁ | | 0.6 | 0.6 | 0.8 | 0.9 | 1.0 | 1.0 |
| ΔG₁ (kcal/mol⁻¹) | | −10.1 | −11.4 | −11.2 | −10.7 | −11.2 | −11.5 |
| ΔH₁ (kcal/mol⁻¹) | | 15.5 | 13.4 | 13.9 | 14.9 | 14.3 | 14.0 |
| ΔS₁ (cal/mol/ | | 82.7 | 79.9 | 80.9 | 82.6 | 82.3 | 82.3 |
| deg⁻¹) | | | | | | | |
| K_{d2} (10⁻⁹ M) | II&III | 1,176.4 | 242.7 | 209.6 | 354.6 | 543.5 | 434.8 |
| n₂ | | 2.4 | 1.6 | 1.5 | 1.5 | 1.5 | 1.4 |
| ΔG₂ (kcal/mol⁻¹) | | −8.4 | −9.4 | −9.5 | −9.1 | −9.1 | −9.0 |
| ΔH₂ (kcal/mol⁻¹) | | 8.3 | 10.1 | 10.1 | 10.9 | 11.3 | 11.7 |
| ΔS₂ (cal/mol/ | | 53.9 | 62.8 | 63.2 | 64.6 | 65.7 | 66.7 |
| deg⁻¹) | | | | | | | |

(These experiments were performed with a 0.5 mM phytate solution at 310 K at each pH adjusted with 0.1 N HCl. n values represent the number of Gd³⁺ bound per phytate molecule. The thermodynamic parameters were provided for analyzing Gd³⁺ binding to the phytate solution.)

### Example 1.2

### ITC Analysis of Phytate with Manganese (Mn²⁺)

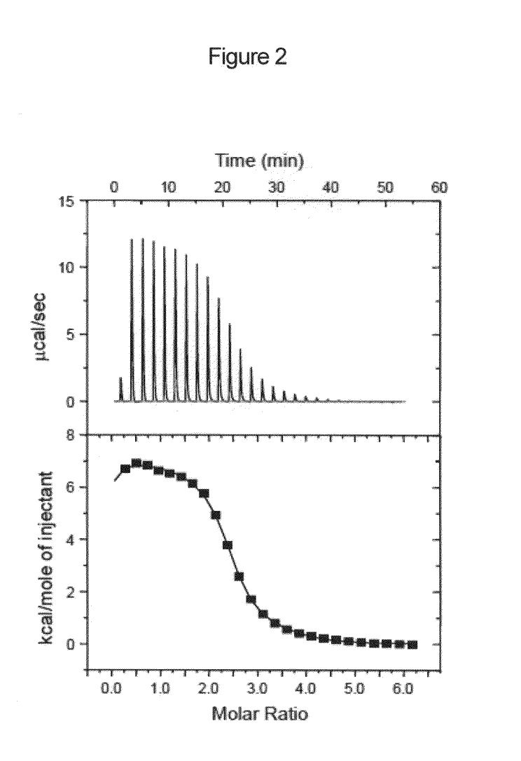

Calorimetric titration of phytate (0.5 mM) with a Mn²⁺ solution (10 mM) was performed at a pH ranging from 3.0 to 8.0 and 37° C. The binding isotherm, which corresponds to a plot of integrated heats as a function of the molar ratio of Mn²/phytate, is depicted in FIG. 2. In this plot, the solid line shows the best-fit curve of the data into the sequential binding sites, as determined mathematically, indicating ligand (Mn²⁺) binding to four different independent sites. From analysis of the data, the dissociation constant (K_{d}), the number of Gd³⁺ bound per phytate molecule (n), and the association enthalpy change (ΔH) were obtained. The thermodynamic parameters from this analysis suggested that 4 mol of Mn²⁺ could bind to one mol of phytate. The binding affinities for Me were 9.52×10⁻⁶ M, 1.1×10⁻⁶ M, 2.21×10⁻⁵ M and 1.2×10⁻⁴ M, respectively. As is apparent from the results of the analysis, Mn²⁺ ion was also found to tightly bind to phytate molecules.

### Example 1.3

### ITC Analysis of Phytate with Ca²⁺ and Mg²⁺

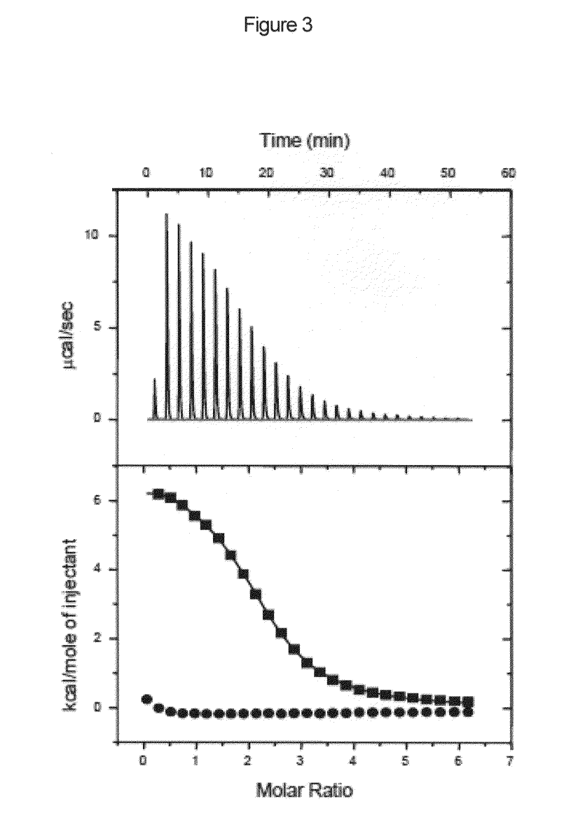

Since Ca²⁺ and Me ions are the most abundant mineral ions in the blood, it is very important to compare the relative binding affinities of these ions for the phytate with those of various paramagnetic substances. In this regard, calorimetric titration of phytate (0.5 mM) with Ca²⁺ solution (15 mM) or Me solution (30 mM) was performed as a function of pH ranging from 3.0 to 8.0 at 37° C. The binding isotherm which corresponds to a plot of integrated heats as a function of the molar ratio of Ca²⁺/phytate, is depicted in FIG. 3. In this plot, the solid line shows the best-fit curve of the data into the two sets of site models, as determined mathematically, indicating ligand (Ca²⁺) binding to three non-identical independent sites. From data analysis, the dissociation constant (K_{d}), the number of Gd³⁺ bound per phytate molecule (n), and the association enthalpy change (ΔH) were obtained. The thermodynamic parameters corresponding to Ca²⁺ binding to phytate are listed in Table 2.

TABLE 2

| Parameters | Site | pH 2 | pH 3 | pH 4 | pH 5 | pH 6 | pH 7 | pH 8 | pH 9 |
|---|---|---|---|---|---|---|---|---|---|
| K_{d1} (10⁻⁵ M) | I | 328.95 | 19.16 | 1.92 | 2.97 | 6.10 | 3.94 | 8.77 | 2.71 |
| n₁ | | 0.80 | 0.72 | 0.89 | 1.18 | 1.27 | 0.54 | 0.1 | 0.45 |
| ΔG₁ (kcal/mol⁻¹) | | −4.95 | −6.68 | −8.10 | −7.86 | −7.40 | −7.65 | −7.16 | −7.89 |
| ΔH₁ (kcal/mol⁻¹) | | 7.58 | 5.90 | 6.05 | 6.74 | 7.05 | 6.92 | −3.47 | −4.44 |
| ΔS₁ (cal/mol/ | | 40.4 | 40.6 | 45.6 | 47.1 | 46.6 | 47 | 11.9 | 11.1 |
| deg⁻¹) | | | | | | | | | |
| K_{d2} (10⁻⁵ M) | II&III | | 195.31 | 23.53 | 51.28 | 60.61 | 94.34 | 44.84 | 23.31 |
| n₂ | | | 2.73 | 3.11 | 4.24 | 2.01 | 1.84 | 2.52 | 2.77 |
| ΔG₂ (kcal/mol⁻¹) | | | −5.25 | −6.56 | −6.10 | −5.99 | −5.72 | −6.17 | −6.56 |
| ΔH₂ (kcal/mol⁻¹) | | | 2.10 | 3.85 | −2.33 | 7.98 | 5.72 | 6.76 | 7.26 |
| ΔS₂ (cal/mol/ | | | 17.6 | 21.3 | 19.6 | 21.9 | 36.9 | 41.7 | 44.6 |
| deg⁻¹) | | | | | | | | | |

(These experiments were performed with a 0.5 mM phytate solution at 310 K at each pH adjusted with 0.1 N HCl. n values represent the number of Cd²⁺ bound per phytate molecule. The thermodynamic parameters were provided for analyzing Cd²⁺ binding to phytate solution).

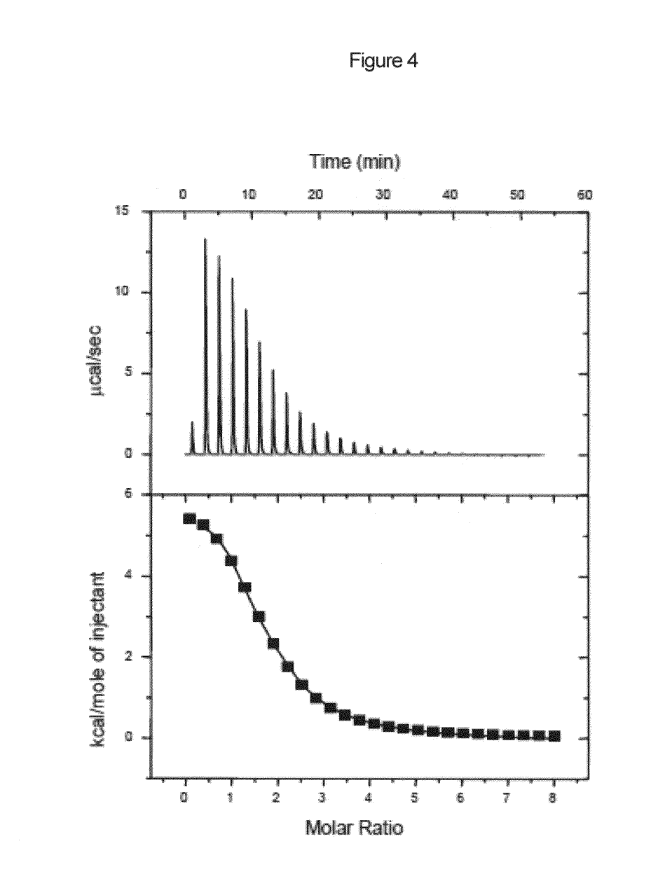

The binding isotherm, which corresponds to a plot of integrated heats as a function of the molar ratio of Mg²/Phytate, is represented in FIG. 4. The solid line shows the best-fit curve of the data into one set of sitemodels, as determined mathematically, indicating ligand (Mg²⁺) binding to one or two identical independent sites. The thermodynamic parameters corresponding to Mg²⁺ binding to phytate are given in Table 3, below. As is apparent from the data, the binding affinity for Ca²⁺ to phytate is 100-fold stronger than that of Mg²⁺.

TABLE 3

| Parameters | pH 2 | pH 3 | pH 4 | pH 5 | pH 6 | pH 7 | pH 8 |
|---|---|---|---|---|---|---|---|
| K_{d1} (10⁻⁴ M) | 2.66 | 1.83 | 1.63 | 1.85 | 1.85 | 1.33 | 1.10 |
| n₁ | 0.71 | 0.881 | 0.966 | 1.59 | 1.59 | 1.66 | 1.93 |
| ΔG₁ (kcal/mol⁻¹) | −5.08 | −5.28 | −5.37 | −5.29 | −5.29 | −5.49 | −5.62 |
| ΔH₁ (kcal/mol⁻¹) | 7.38 | 6.66 | 6.81 | 6.71 | 6.71 | 6.38 | 6.66 |
| ΔS₁ (cal/mol/ | 40.2 | 38.5 | 39.3 | 38.2 | 38.7 | 38.3 | 39.6 |
| deg⁻¹) | | | | | | | |

(These experiments were performed with a 0.5 mM phytate solution at 310 K at each pH adjusted with 0.1 N HCl. n values represent the number of Mg²⁺ bound per phytate molecule. The thermodynamic parameters were provided for analyzing Me binding to phytate solution.)

From the ITC analysis of phytate with various metal ions, it was found that Gd³⁺ most tightly binds with the phytate even at acidic pH values, indicating that these complexes are thermodynamically the most stable and show kinetic inertness with respect to metal loss at acidic pH. This enables Gd-phytate complexes to be used as contrast agents which are low in toxicity.

### Example 1.4

### ITC Analysis for Binding Thermodynamics of Gd³⁺ to Inositol Phosphates

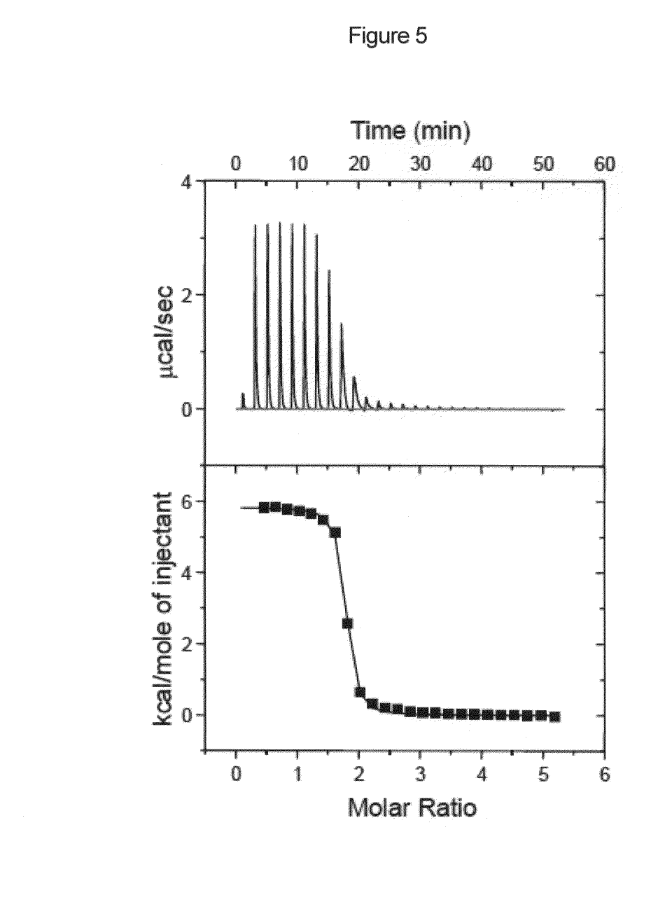

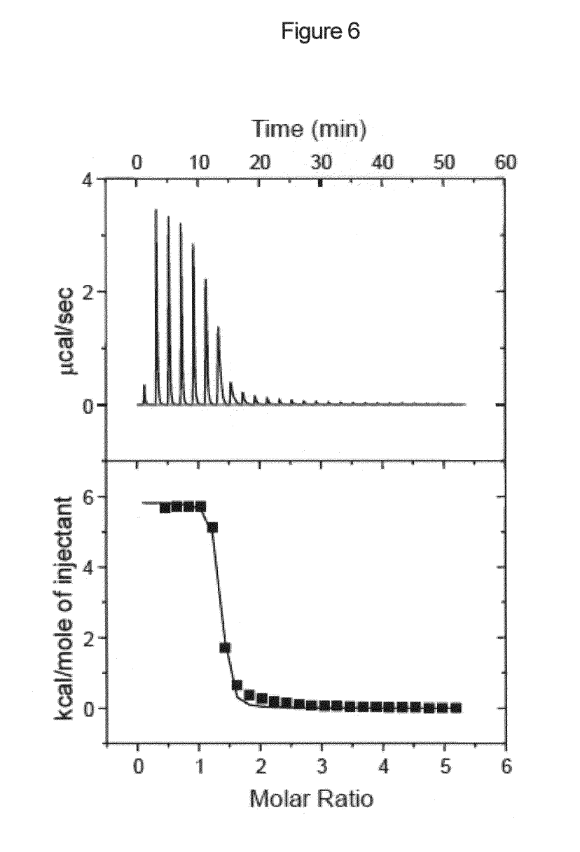

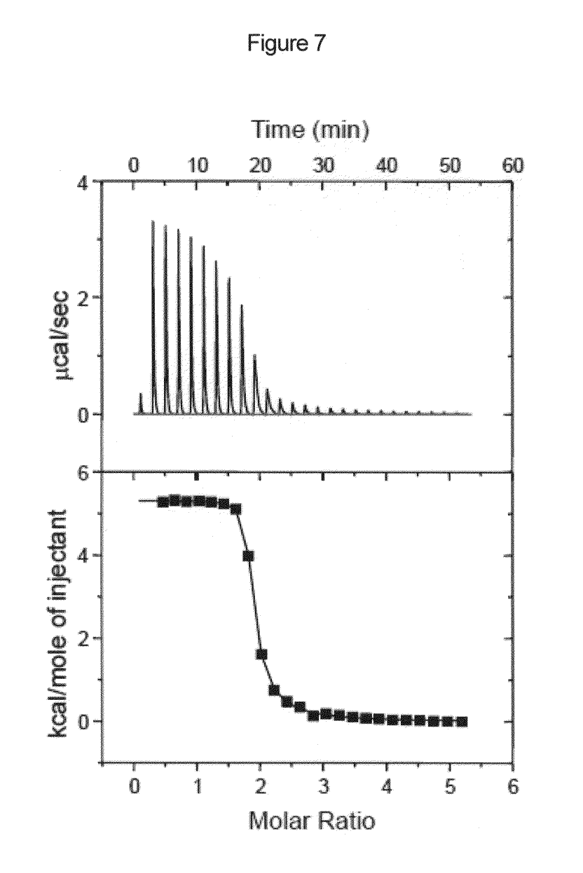

Calorimetric titration of inositol phosphates (inositol-1,3,4-trisphosphate, inositol-1,4,5-trisphosphate, or inositol-1,3,4,5-tetrakisphosphate) (0.2 mM) with Gd³⁺ (5 mM) was performed at 25° C., 10 mM HEPES pH 7.0. The binding isotherm, which corresponds to a plot of integrated heats as a function of the molar ratio of Gd³⁺/inositol phosphate is represented in FIGS. 5, 6 and 7, respectively. The thermodynamic parameters corresponding to Gd³⁺ binding per mole of the inositol phosphate derivatives are summarized in Table 4, below. The results indicate that inositol phosphates also have strong binding affinities for

TABLE 4

| | | Inositol | Inositol- | Inositol- |
|---|---|---|---|---|
| | | 1,3,4- | 1,4,5- | 1,3,4,5- |
| Parameters | Site | tisphosphate | tisphosphate | tetraphosphate |
| K_{d1} (10⁻⁷ M) | I | 7.19 | 5.61 | 2.88 |
| n₁ | | 1.71 | 1.28 | 1.97 |
| ΔG₁ (kcal/mol⁻¹) | | −8.93 | −9.10 | −9.47 |
| ΔH₁ (kcal/mol⁻¹) | | 5.83 | 5.84 | 5.32 |
| ΔS₁ (cal/mol/deg⁻¹) | | 47.6 | 48.2 | 47.7 |
| K_{d2} (10⁻⁵ M) | II | | | 69.44 |
| n₂ | | | | 0.11 |
| ΔG₂ (kcal/mol⁻¹) | | | | −6.22 |
| ΔH₂ (kcal/mol⁻¹) | | | | 8.51 |
| ΔS₂ (cal/mol/deg⁻¹) | | | | 47.5 |

(These experiments were performed with a 0.2 mM phytate solution at 298 K, 10 mM HEPES, and a pH of 7.0. n values represent the number of Gd³⁺ bound per phytate molecule. The thermodynamic parameters were provided for analyzing Gd³⁺ binding to phytate solution.)

### Example 1.5

### ITC Analysis for Binding Thermodynamics of Fe³⁺ to Phytate

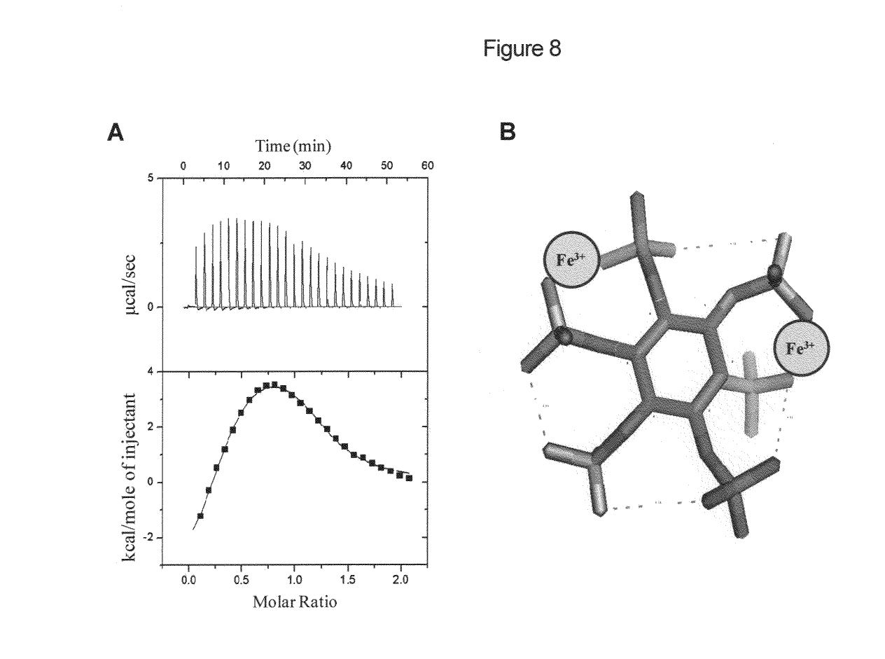

Calorimetric titration of phytate (0.5 mM) with a Fe³⁺ solution (5 mM) was measured as a function of pH ranging from 4.0 to 6.0 at 37° C. The binding isotherm, which corresponds to a plot of integrated heats as a function of the molar ratio of Fe³⁺/Phytate, is represented in FIG. 8A. In this figure, the solid line corresponds to the best-fit curve of the data between the phytate and iron ion into the two different sets of site models, as mathematically determined, indicating ligand (Fe³⁺ binding to two non-identical independent sites. From analysis of the data, the dissociation constant (K_{d}), the number of Fe³⁺ bound per phytate molecule (n), and the association enthalpy change (ΔH) were obtained. The thermodynamic parameters corresponding to Fe³⁺ binding to phytate solution are given in Table 5. As is apparent from the data of Table 1, Fe³⁺ ions strongly bind two oxianions from the phosphate groups of phytate in a bidentate fashion (FIG. 8B) to form Fe-phytate complexes with high affinity of 10⁻⁶-10⁻⁵ M.

TABLE 5

| Parameters | Site | pH 4 | pH 5 | pH 6 |
|---|---|---|---|---|
| K_{d1} (10⁻⁶ M) | I | 54.35 | 1.62 | 4.61 |
| n₁ | | 0.72 | 0.16 | 0.35 |
| ΔG₁ (kcal/mol⁻¹) | | −6.06 | −8.20 | −7.57 |
| ΔH₁ (kcal/mol⁻¹) | | 3.58 | −0.0466 | −5.24 |
| ΔS₁ (cal/mol/deg⁻¹) | | 31.1 | 26.3 | 7.54 |
| K_{d2} (10⁻⁵ M) | II | | 4.10 | 3.13 |
| n₂ | | | 0.88 | 0.88 |
| ΔG₂ (kcal/mol⁻¹) | | | −6.21 | −6.38 |
| ΔH₂ (kcal/mol⁻¹) | | | −4.12 | 5.93 |
| ΔS₂ (cal/mol/deg⁻¹) | | | 33.3 | 39.7 |

(These experiments were performed with a 0.5 mM phytate solution at 310 K at each pH adjusted with 0.1 N HCl. n values represent the number of Fe³′ bound per phytate molecule. The thermodynamic parameters were provided for analyzing Fe³⁺ binding to the phytate solution.)

### Example 2

### ITC Analysis for Binding Thermodynamics of Gd³⁺ to EDTA Solution

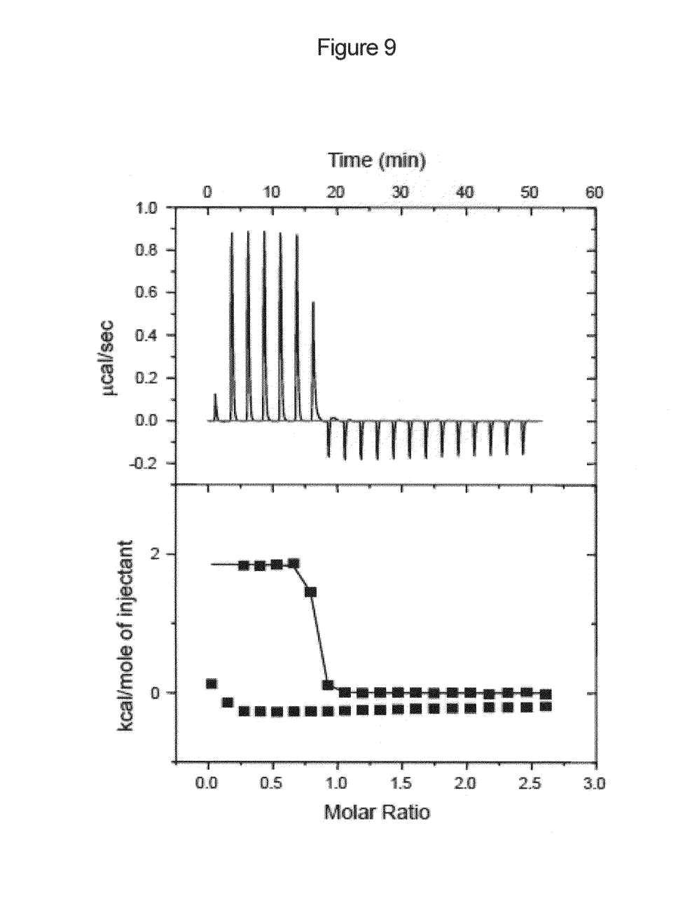

Calorimetric titration of EDTA (0.4 mM) with Gd³⁺ solution (5 mM) was performed at 25° C. in 10 mM MES at a pH of 5.6. The binding isotherm, which corresponds to a plot of integrated heat as a function of the molar ratio of Gd³⁺/EDTA, is depicted in FIG. 9. In this plot, the solid line corresponds to the best-fit curve of the data into one set of site models, as determined mathematically, indicating one ligand (Gd³⁺) binding to one EDTA molecule. From data analysis, the dissociation constant (K_{d}), the number of Gd³⁺ bound per EDTA molecule (n), and the association enthalpy change (ΔH) were obtained. The dissociation constant (K_{d}) was determined to be 1.47×10⁻⁷ M. This is an important practical approach to determining the relative stability compared to the known chelating agent EDTA. Also, the results suggested that the binding affinity for Gd³⁺ binds to phytate with a 10-fold higher affinity than to EDTA, and thus the contrast agents of the present invention (Gd-phytate complexes) are 10-fold more stable than are Gd-EDTA.

### Example 3

### ITC Analysis for Binding Thermodynamics of Gd³⁺ to Diethylene Triamine Pentaacetic Acid (DTPA)

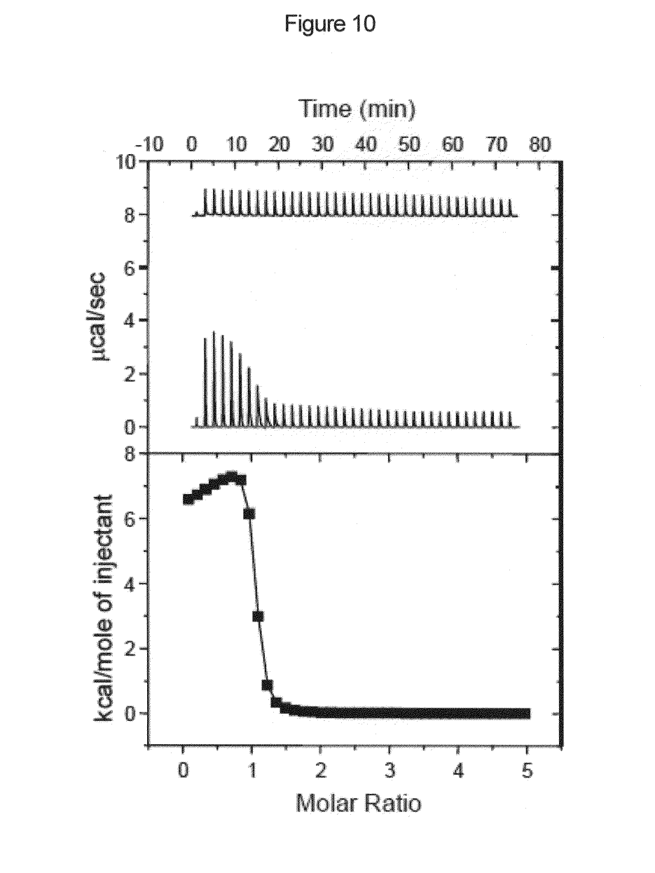

Calorimetric titration of DTAP (0.2 mM) with Gd³⁺ solution (5 mM) was performed in 10 mM sodium acetate, pH 4.8 or in 10 mM HEPES, pH 7.0 at 25° C. in order to determine the relative stability compared to that of Gd-phytate complexes. The binding isotherm, which corresponds to a plot of integrated heat as a function of the molar ratio of Gd³⁺/DTPA, is represented in FIGS. 10 (pH 4.8) and 11 (pH 7.0). As determined mathematically, the solid line shows the best-fit curve of the data into the two sets of site models, which described ligand (Gd³⁺) binding to two non-identical independent sites. From data analysis, the dissociation constant (K_{d}), the number of Gd³⁺ bound per EDTA molecule (n), and the association enthalpy change (ΔH) were obtained. The thermodynamic parameters corresponding to Gd³⁺ binding to DTPA are summarized in Table 6, below. As seen in this table, the binding affinities for Gd³⁺ to phytate are 10-˜100-fold higher than those of Gd³⁺ to DTPA, suggesting that Gd-phytate complexes are more kinetically inert than those of Gd-DTPA.

TABLE 6

| Parameters | Site | pH 4.8 | pH 7.8 |
|---|---|---|---|
| K_{d1} (10⁻⁷ M) | I | 7.14 | 6.37 |
| n₁ | | 0.68 | 0.7 |
| ΔG₁ (kcal/mol⁻¹) | | −8.81 | −8.72 |
| ΔH₁ (kcal/mol⁻¹) | | 2.23 | −1.84 |
| ΔS₁ (cal/mol/deg⁻¹) | | 35.6 | 22.2 |
| K_{d2} (10⁻⁷ M) | II | 9.0 | 181.16 |
| n₂ | | 0.32 | 0.9 |
| ΔG₂ (kcal/mol⁻¹) | | −9.30 | −6.94 |
| ΔH₂ (kcal/mol⁻¹) | | 18.10 | 5.27 |
| ΔS₂ (cal/mol/deg⁻¹) | | 88.4 | 39.4 |

(These experiments were performed with 0.2 mM DTPA in 10 mM sodium acetate, pH 4.8 or in 10 mM HEPES, pH 7.8 at 298 K. n values represent the number of Gd³⁺ bound per mole of inositol phytate. The thermodynamic parameters were provided for analyzing Gd³⁺ binding to phytate solution.)

### Example 4

### Preparation of Paramagnetic-Phytate Complexes

So long as it is paramagnetic, any element, molecule, ion or compound might be used to prepare paramagnetic-phytate complexes. A paramagnetic substance includes at least one of the following elements: the ion of transition elements such as Cr³⁺, Co²⁺, Mn²⁺, Ni²⁺, Fe²⁺, Fe³⁺, Cu²⁺, and Cu³⁺; or the ion of lanthanide elements such as La³⁺, Ce³⁺, Tb³⁺, Pr³⁺, Dy³⁺, Nd³⁺, Ho³⁺, Pm³⁺, Er³⁺, Sm³⁺, Tm³⁺, Eu³⁺, Yb³⁺, and Lu³⁺, with a preference for Fe²⁺, Fe³⁺, Mn²⁺ or Gd³⁺. Gd³⁺ is most preferred since it has the strongest paramagnetic property. However, Gd is very expensive and highly toxic in its free ion state under physiological conditions. The chelation of Gd with phytate, a strong chelator, would produce a physiologically tolerable and stable form of Gd which is suitable for use in a contrast agent.

### Example 4.1

### Preparation of Gd-Phytate Complexes

The binding affinity of phytate for Gd³⁺ ions was found to be the strongest at an equimolar concentration of Gd³⁺ and phytate as measured by ITC. On the basis of these ITC studies (FIG. 1, Table 1), a Gd-phytate solution (20 mM) was prepared using equimolar concentrations of Gd³⁺ and phytate. In this regard, 7.24 g of sodium phytate (Sigma chemical Co., USA) was dissolved in 300 mL of sterile water, adjusted to a pH of 7.0 with 1 N hydrochloric acid solution, and mixed with 100 mL of a 200 mM GdCl₃ solution (Sigma chemical Co., USA) to give 1,000 mL of a Gd-phytate solution. This solution was then aliquoted at doses of 10 mL into 15 mL vials. The pH of the aqueous solution might range from 4 to 9, most preferably from 6 to 8 when this contrast agent was used diagnostically under physiological conditions. Gd-phytate complexes could be prepared in any molar ratio of Gd³⁺ and phytate in the range from 0.5 to 3 or vice versa. The most preferred concentration was an equimolar concentration of Gd³⁺ and phytate. Ca²⁺ ions might be added to Gd-phytate complexes so as to minimize Ca²⁺ chelation of Gd-phytate complexes from the blood vessels when Gd-phytate complexes are used diagnostically. The most preferred Ca²⁺ concentration is an equimolar concentration of Gd³⁺ and phytate.

### Example 4.2

### Preparation of Mn-Phytate Complexes

ITC analysis suggested that the binding affinity of phytate for Mn²⁺ ions was most strong at equimolar concentrations of Mn²⁺ and phytate. On the basis of these results (FIG. 2), a Mn-Phytate solution (20 mM) was prepared using equimolar concentrations of Me and phytate. To this end, 7.24 g of sodium phytate (Sigma chemical Co., USA) was dissolved in 300 mL of sterile water, adjusted to pH 7.0 with 1 N hydrochloric acid solution, and mixed with 100 mL of 200 mM MnCl₂ solution (Sigma chemical Co., USA) to give 1000 mL of a Mn-phytate solution. Then, this solution was aliquoted at doses of 10 mL to 15 mL vials. The pH of the aqueous solution might range from 4 to 9, most preferably from 6 to 8 when this contrast agent was used diagnostically under physiological conditions. Mn-phytate complexes could be prepared at any molar ratio of Mn²⁺ and phytate in the range from 0.5 to 4 or vice versa. The most preferred concentration is an equimolar concentration of Mn²′ and phytate. Ca²⁺ ions might be added to Md-phytate complexes so as to minimize Ca²⁺ chelation of Md-phytate complexes from the blood vessels when Md-phytate complexes are used diagnostically. The most preferred Ca²⁺ concentration is an equimolar concentration of Me and phytate.

### Example 4.3

### Preparation of Fe-Phytate Complexes

The binding affinity of phytate for Fe³⁺ ions was found to be the strongest at an equimolar concentration of Fe²⁺: and phytate as measured by ITC. On the basis of these ITC studies (FIG. 8, Table 5), a Fe-phytate solution (20 mM) was prepared at equimolar concentrations of Fe³⁺ and phytate. In this regard, 7.24 g of sodium phytate (Sigma chemical Co., USA) was dissolved in 300 mL of sterile water, the pH of which were adjusted to 6.0 with 1 N hydrochloric acid solution, and mixed with 100 mL of a 200 mM FeCl₃ solution (Sigma chemical Co., USA) to give 1,000 mL of a Gd-phytate solution. This solution was then aliquoted at doses of 10 mL into 15 mL vials. The pH of the aqueous solution might range from 4 to 9, most preferably from 6 to 8 when this contrast agent was used diagnostically under physiological conditions. Fe-phytate complexes could be prepared in any molar ratio of Fe³⁺ and phytate in the range from 0.5 to 3 or vice versa. The most preferred concentration was an equimolar concentration of Fe³⁺ and phytate. Ca²⁺ ions might be added to Fe-phytate complexes so as to minimize Ca²⁺ chelation of Fe-phytate complexes from the blood vessels when Fe-phytate complexes are used diagnostically. The most preferred Ca²⁺ concentration is an equimolar concentration of Fe³⁺ and phytate.

### Example 5

### Preparation of Paramagnetic-Inositol Phosphates

So long as it is paramagnetic, any element, molecule, ion or compound may be used to prepare paramagnetic-inositol phosphate complexes. The paramagnetic substance useful in the present invention includes at least one of the following: ions of transition elements such as Cr³⁺, Co²⁺, Mn²⁺, Ni²⁺, Fe²⁺, Fe³⁺, Cu²⁺, and Cu³⁺; and ions of lanthanide elements such as La³⁺, Gd³⁺, Ce³⁺, Tb³⁺, Pr³⁺, Dy³⁺, Nd³⁺, Ho³⁺, Pm³⁺, Er³⁺, Sm³⁺, Tm³⁺, Eu³⁺, Yb³⁺, and Lu³⁺, with a preference for Fe²⁺, Fe³⁺, Mn²⁺ or Gd³⁺. Gd³⁺ is most preferred since it has the strongest paramagnetic property.

The contrast agent based on inositol phosphates may comprise any of d-myo-inositol-1,2,3,4,5-pentaphophate, d-myo-inositol-1,3,4,5-tetraphophate, d-myo-inositol-1,4,5-trisphosphate, and d-myo-inositol-1,3,4-trisphosphate.

### Example 5.1

### Preparation of Gd-Inositol Phosphate Complexes

The binding affinity of inositol phosphates (d-myo-inositol-1,3,4-trisphosphate, d-myo-inositol-1,4,5-trisphosphate, or d-myo-inositol-1,3,4,5-tetrakisphosphate) for Gd³⁺ ions was found to be the strongest as measured by ITC. On the basis of these results (FIGS. 5, 6 and 7, Table 4), Gd-inositol phosphates were prepared using an equimolar concentration of Gd³⁺ and inositol phosphate. In this regard, 20 mM of inositol phosphate (Sigma chemical Co., USA) was dissolved in 5 mL of sterile water, adjusted to pH 7.0 with 1 N hydrochloric acid solution, and mixed with 5 mL of a 20 mM Gd³⁺ solution (Sigma chemical Co., USA) to give an aqueous solution of Gd-inositol phosphate. The pH of the aqueous solution might range from 4 to 9, most preferably from 6 to 8 when this contrast agent was used diagnostically under physiological conditions. Gd-inositol phosphates could be prepared in any molar ratio of Gd³⁺ and inositol phosphate in a range from 0.5 to 4 or vice versa. The most preferred concentration was an equimolar concentration of Gd and inositol phosphate.

### Example 6

### Preparation Paramagnetic-Phosphatidylinositol Phosphate Liposomes

So long as it is paramagnetic, any element, molecule, ion or compound may be used to prepare paramagnetic-phosphatidylinositol phosphate liposomes. The paramagnetic substance useful in the present invention includes at least one of the following: ions of transition elements Cr³⁺, Co²⁺, Mn²⁺, Ni²⁺, Fe²⁺, Fe³⁺, Cu²⁺, and Cu³⁺; and ions of lanthanide elements such as La³⁺, Gd³⁺, Ce³⁺, Tb³⁺, Pr³⁺, Dy³⁺, Ho³⁺, Pm³⁺, Er³⁺, Sm³⁺, Tm³⁺, Eu³⁺, Yb³⁺, and Lu³⁺, with a preference for Fe²⁺, Fe³⁺, Mn²⁺ or Gd³⁺. Gd³⁺ is most preferred since it has the strongest paramagnetic property.

The contrast agent based on phosphatidylinositol phosphate derivatives may comprise any of and phosphatidylinositol-4,5-bisphosphate.

### Example 7

### Measurement of Stability Constant (K) of Gd-Phytate, Gd-DTAP, Gd-DOTA, and Gd-EDTA from Thermodynamic Parameters of ITC Analysis

A stability constant (formation constant, binding constant) is an equilibrium constant for the formation of a complex in solution. It is a measure of the strength of the interaction between the reagents that come together to form the complex. The thermodynamics of metal ion complex formation provides much significant information. The chelate effect, below, is best explained in terms of thermodynamics. An equilibrium constant is related to the standard Gibbs free energy change for the reaction.

ΔG°=−2.303 RT log 10 K, where R is the gas constant and T is the temperature in Kelvin. At 25° C., ΔG in kJ mol⁻¹ equals to 5.708 log K (1 kJ mol⁻¹=1000 Joules per mole). From this equation, the stability constant (K) were calculated and listed in Table 7. The stability constant for Gd-phytate was much higher than those of other Gd-complexes. This indicated that Gd-phytate is highly stable at the physiological conditions, resulting in a high thermodynamic stability and kinetic inertness with respect to metal loss.

TABLE 7

| Contrast agents | Stability Constant (K) |
|---|---|
| Gd-phytate | 1.9 × 10³⁵ M⁻¹ |
| Gd-DTPA | 1.2 × 10²⁸ M⁻¹ |
| Gd-DOTA | 1.3 × 10²⁹ M⁻¹ |
| Gd-EDTA | 4.4 × 10²⁹ M⁻¹ |

### Example 8

### Measurement of Relaxation Rates (R₁) of Mn²⁺, Gd-DTPA, Mn-Phytate and Gd-Phytate Complexes at Different Concentrations in a 9.4 T MRI Scanner

In contrast-enhanced MRI using a contrast agent, relaxation rate (R1=1/T1) is a measure of how efficiently the contrast agent perturbs a local magnetic field to produce an image contrast. In this context, the relaxation rates of Mn-phytate and Gd-phytate were estimated at different concentrations to be compared to those of Mn²⁺ and Gd-DTPA, which were widely utilized in MR research.

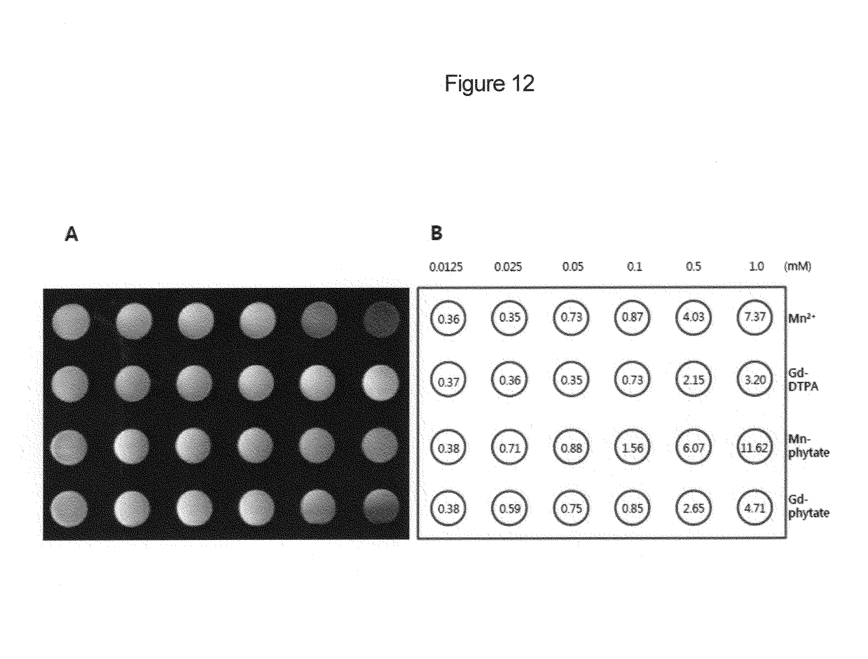

A total of six tube phantoms were made for each of the four chemical compounds (Mn²⁺, Gd-DTPA, Mn-phytate and Gd-phytate) at concentrations of 0.0125, 0.025, 0.05, 0.1, 0.5 and 1 mM. The experiments were conducted on a 9.4 T Bruker biospec MRI scanner equipped with a volume coil (7 cm in diameter) for both transmission and reception. T1 was estimated by using a spoiled gradient echo sequence (SPGR) with 11 inversion recovery times (T1's=16, 20, 30, 50, 100, 200, 400, 700, 1500, 3000 and 6000 ms). The image obtained at T1=3000 ms is shown in FIG. 12A. Other sequence parameters were as follows: TR/TE=8000/4.41 ms, flip angle (FA)=90°, field of (FOV)=60×40 mm, matrix size=256×256, 1 slice with a thickness of 1 mm, 2 signal averages.

As shown in FIG. 12, both Mn-phytate and Gd-phytate produced higher contrast effects than did Me and Gd-DTPA at all concentrations.

### Example 9

### MR Signal Change as a Function of the Concentrations of Gd-Phytate in RAW 264.7 Macrophage Cells in a 9.4 T Scanner

This example attempts to demonstrate the phagocytosis of Gd-phytate by macrophages and the image contrast enhancement as a function of the concentrations of the agent.

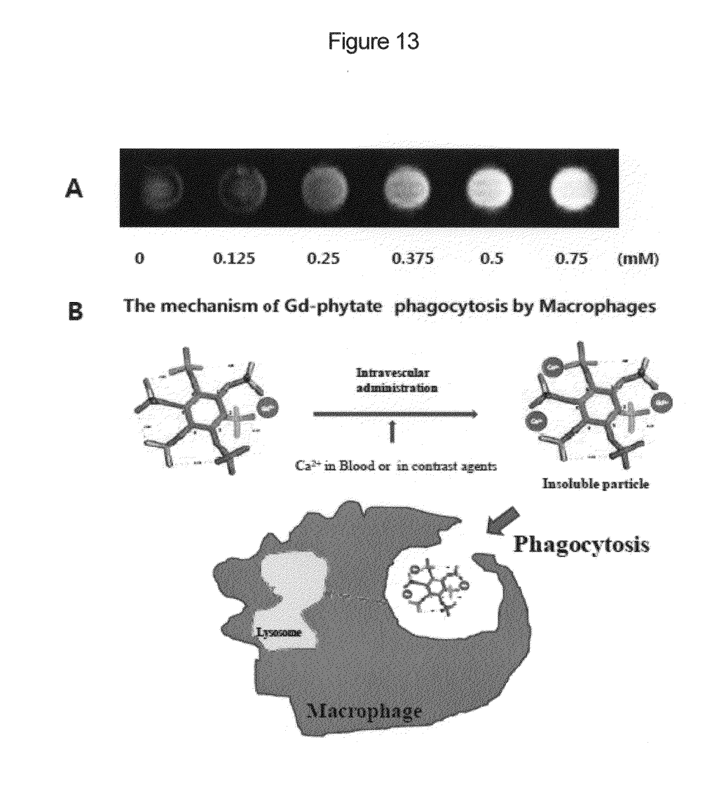

RAW 264.7 macrophages were purchased from the American Type Culture Collection (Manassas, Va., USA). Cells were cultured in complete DMEM supplemented with 10% FBS, 1% penicillin-streptomycin, 1% glutamine and 1% sodium pyruvate at 37° C. in a 5% CO₂ incubator. For MR imaging experiments, the cells were plated at a density of 1×10⁵ cells/well in 6-well plates. After incubation for 24 hrs, the cells were washed with PBS. Thereafter, each well was incubated for 3 hrs with Gd-phytate at different concentrations (0, 0.125, 0.25, 0.375, 0.5 and 0.75 mM), followed by washing three times with PBS. The cell pellets were suspended in 1 mL of PBS and transferred to 0.2 mL microtubes. Using a 9.4 T Bruker biospec MRI scanner equipped with a volume coil (7 cm in diameter) for both transmission and reception, T1-weighted images were obtained. The T1-weighted image was acquired with SPGR and is shown in FIG. 13A. The sequence parameters are: TR/TE 12.8/1.4 ms, FA=90°, 1 slice with a thickness of 1 mm, 32 signal averages.

This example demonstrates the MR characteristic of the proposed Gd-phytate as a positive contrast agent, that is, a T1 contrast agent, and the uptake of the Gd-complex by macrophages. The T1-weighted signal intensity of RAW 264.7 macrophage cell line was increased with increasing Gd-phytate concentration. Hence, the contrast agent of the present invention could potentially visualize the tissue characteristics of a targeted region of interest in vivo by the mechanism illustrated in FIG. 13B. That is, after intravenous administration, Gd-phytate and Mn-phytate complexes were selectively ingested by macrophages, which are located in the organs of the mononuclear phagocyte system (i.e. the liver, the spleen, the bone marrow, lymph nodes), suggesting that Kupffer cells from blood vessels directly uptake Gd-phytate complexes. Once taken in by hepatic Kupffer cells, the contrast agents of the present invention exhibited high T1 and T2 relaxivity, generating distinct microscopic field inhomogeneities.

### Example 10

### Time-Dependent Changes in T1-Weighted MR Signal in Rat Liver after Intravenous Administration of Mn-phytate Complex at 9.4 T

This example attempts an in vivo demonstration of the MR characteristic of the Mn-complex as a positive contrast agent and the phagocytosis of Mn-phytate by Kupffer cells.

A 20 mM solution of Mn-phytate (equimolar concentrations of Mn²⁺ and phytate) was administered at a dose of 5 mmol/kg to the tail vein of a male Sprague Dawley (SD) rat (wt.=300 g) anesthetized with isoflurane. The rat was placed prone at the isocenter of the MIR scanner (9.4 T Broker BioSpec) equipped with a volume coil (7 cm in diameter) for transmission and reception, and T1-weighted images of the liver were obtained by using SPGR in combination with respiratory gating. The sequence parameters are: TR/TE=3.52/1.56 ms, FA=90°, FOV=70×58, matrix size=256×256, 3 slices with a thickness of 1 mm, 32 signal averages.

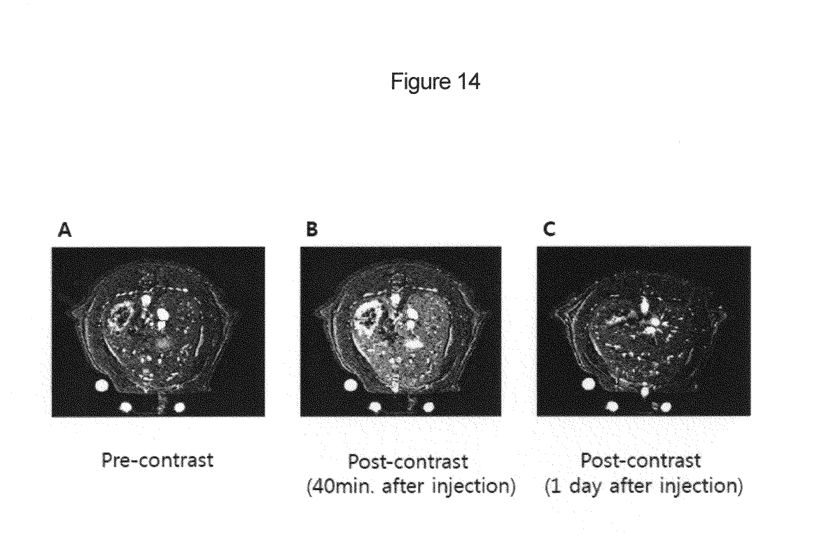

The images are shown in FIG. 14A, confirming that the Mn-phytate functions as a positive contrast agent, that is, a T1 contrast agent. In general, the currently used extracellular MR contrast agents (e.g., Gd-DTPA) are known to greatly reduce in liver concentration within a few minutes after injection. In other words, the conventional contrast agents guarantee contrast effects only for a short period of time. In contrast, the changes in the signal intensity of the liver reach a maximum (up to about 32% enhancement) about 40 minutes after the administration of Mn-phytate. The agent then appears to be almost removed from the liver in about 24 hrs which is significantly shorter than that for other intracellular agents reported to date (e.g., SPIO).

### Example 11

### Time-Dependent Changes in T1-Weighted MR Signal in Rat Liver after Intravenous Administration of Gd-Phytate Complex at 9.4 T

This example is intended to provide an in vivo demonstration of the MR characteristic of the Gd-complex as a positive contrast agent, that is, a T1 agent and the phagocytosis of Gd-phytate by Kupffer cells in the liver.

A 20 mM solution of Gd-phytate (equimolar concentrations of Gd³⁺ and phytate) was administered at a dose of 4 μmol/kg to the tail vein of a male Sprague Dawley (SD) rat (wt.=300 g). The rat was placed prone at the isocenter of the magnet (9.4 T Bruker BioSpec) equipped with a volume coil (7 cm in diameter) for both transmission and reception, and T2-weighted images of the liver were obtained using a fast spin echo (FSE) in combination with respiratory gating. In a preliminary experiment (data not shown), the R2 (transverse relaxivity) of Gd-phytate complex dominated R1 (longitudinal relaxivity) at this high field (9.4 T), providing the choice of the T2-weighting FSE rather than the T1-weighting SPGR. The MR characteristic of the agent as a positive contrast, that is, a T1 contrast agent, is demonstrated at lower fields (1.5 and 4.7 T) in the following examples.

The sequence parameters are: TR/TE=6000/12.8 ms, FA=90°/180°, FOV=65×55, matrix size=256×192, 3 slices with a thickness of 1 mm, 2 signal averages.

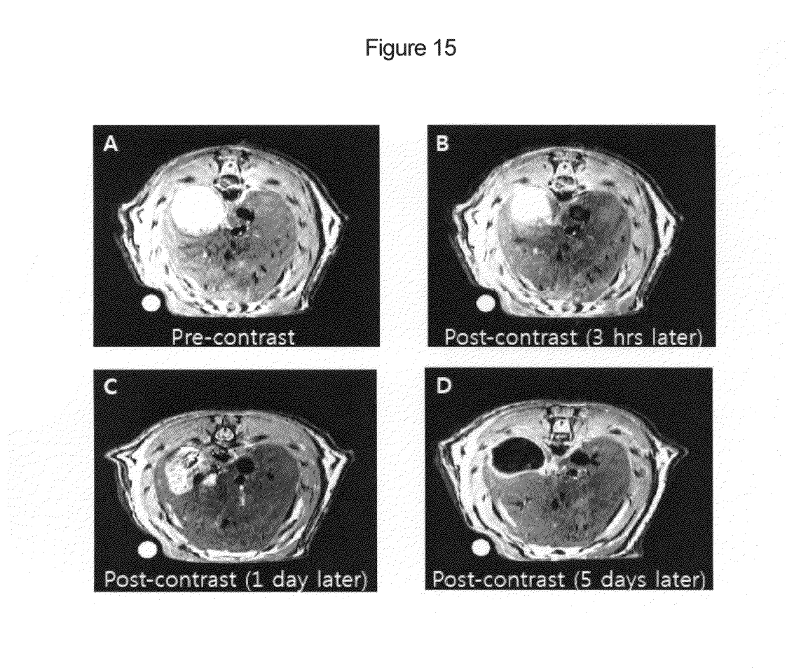

The images are shown in FIG. 15. As can be seen, changes in the signal intensity of the liver reached a maximum (up to about 26% reduction at 4 mmol/kg) about 24 hrs after the administration of Gd-phytate (FIG. 15C). The signal intensity of the liver tissue was observed to recover to its baseline about 5 days after the administration (FIG. 15D), further supporting that the signal changes resulted from the phagocytosis of Gd-phytate by Kupffer cells rather than monotonic elimination as with other extracellular agents.

That is, the Gd-phytate slowly produced an increase in T2-weighted signal intensity of the liver by gradually accumulating over time. Furthermore, the Gd-phytate complex would be very useful clinically as an MR imaging contrast agent that can be taken up by a normal organ after intravenous or subcutaneous injection. Guaranteeing MR imaging to depict in vivo phagocytosis thereof by macrophages, the Gd-phytate complexes find applications in the diagnosis of various diseases such as atherosclerotic plaques, organ graft rejection, multiple sclerosis, and the sentinel lymph node detection of various types of cancer.

### Example 12

### Time-Dependent Changes in T1-Weighted MR Signal in Rat Liver after Intravenous Administration of Gd-phytate Complex as a Positive Contrast Agent at 1.5 and 4.7 T

In order to demonstrate the function of Gd-phytate complex as a positive contrast agent in low magnetic fields, T1-weighted images were obtained using SPGR at lower fields of 1.5 T (Siemens Avanto system) and 4.7 T (Bruker Biospec system).

The sequence parameters are: TR/TE=400/8 ms, FA=90°, FOV=70×70, matrix size=256×256, 11 slices with a thickness of 3 mm, 3 signal averages for 1.5 T, and TR/TE=3.3/1.2 ms, FA=90°, FOV=70×70, matrix size=192×128, 3 slices with a thickness of 1 mm, 32 signal averages for 4.7 T.

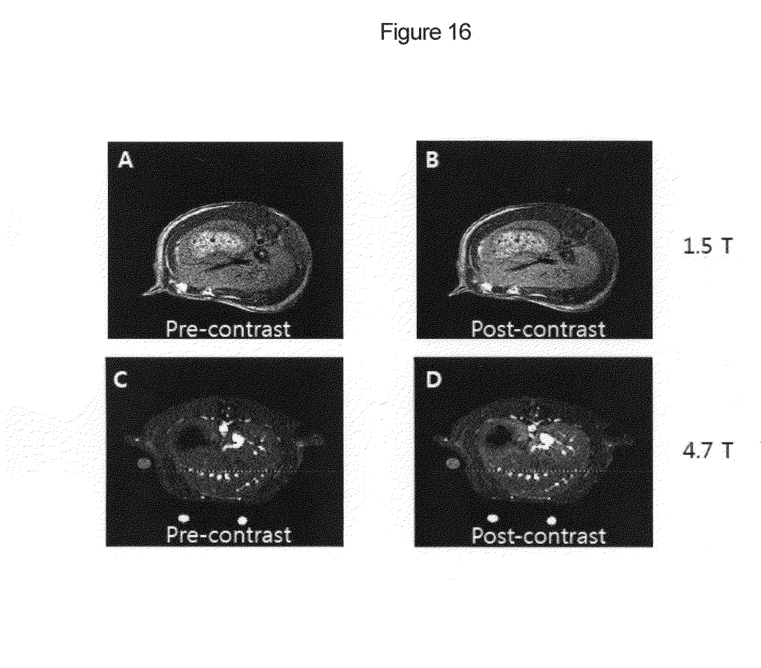

The images are shown in FIG. 16. As seen in this figure, the administration of Gd-phytate at a dose of 4 μmol/kg predominantly shortened the T1 of a normal liver, increasing the MR signal intensity at both 1.5 and 4.7 T. This suggests that the intravenously administered Gd-phytate complexes are taken up by Kupffer cells from blood vessels. Once absorbed by Kupffer cells, the Gd-phytate complexes exhibited high T1 relaxivity and a high magnetic moment, which generates microscopic field inhomogeneities at these lower magnetic fields.

### Example 13

### Fe-phytate Complex as a Novel MRI Contrast Agent for the Detection of Sentinel/Metastatic Lymph Node in C57BL/6 Mice Injected with B16F1/B16F1-GFP Melanoma Cancer Cells at a 9.4 T Scanner.

This example attempts to demonstrate the detection of sentinel/metastatic lymph node through subcutaneous injection of Fe-phytate complex as a MRI contrast agent AT primary tumor of melanoma cancer mice model.

Male C57BL/6 mice (weight 25-30 g, 6-7 weeks old) were purchased from the Orient Bio (Seoul, South Korea) and inbred at the animal facility of Lee Gil Ya Cancer and Diabetes Institute (LCDI), Gachon University of Medicine and Science.

B16F1 melanoma cancer cells were purchased from the American Type Culture Collection (Manassas, Va., USA). For B16F1-GFP cancer cells were transfected to express green fluorescent protein, which can serve as a tumor marker. Cells were cultured in complete DMEM supplemented with 10% FBS, 1% penicillin-streptomycin, 1% glutamine and 1% sodium pyruvate at 37° C. in a 5% CO₂ incubator.

For melanoma cancer model, cancer cells were washed, counted and suspended in culture media for injection. Animals were anesthetized with 4% isoflurane in the induction chamber. Following anesthesia, about 4˜5×10⁵ cells in 20 μl of cell culture media were injected to male C57BL/6 mice subcutaneously into the wrist of left forelimb. The growth of tumor in the mice was monitored daily. MR imaging was performed 13-18 days after the injection of B16F1 or B16F1-GFP cells when the size of grown primary tumor reached about 1 cm.

The experiments for sentinel lymph nodes were performed on a 9.4 T Bruker animal MR scanner (Biospec 94/20 USR; Bruker BioSpin, Ettlingen, Germany). A transmit-only volume coil was used for excitation (Bruker, Ettlingen, Germany). For signal reception a dedicated mouse brain surface coil (Bruker, Ettlingen, Germany) was used.

Mice were anesthetized initially with 4% isoflurane in the induction chamber and then placed on the animal bed (Bruker, Ettlingen, Germany) in a supine position. Mice were then anesthetized by 1.5-2.0% isoflurane inhalation through a nose cone thereafter. To stabilize the body temperature of the mice an animal warming system (Bruker, Ettlingen, Germany) was used, which consists of a warm water (39° C.) reservoir with a pump and hoses that were run underneath the animal bed. An MR-compatible animal monitoring and gating system (SA instrument, USA) was used throughout the experiments.

Transverse, coronal and sagittal scout images were acquired by using a two-dimensional (2D) spoiled gradient echo sequence (SPGR). For lymph node imaging fat-suppressed, respiratory-gated T1-weighted axial images were acquired by using SPGR. The sequence parameters were; repetition time (TR)=335′ ms, echo time (TE)=6.7 ms, flip angle (FA)=90°, matrix size=384×384, number of excitation (NEX)=8. Fat-suppressed, respiratory-gated T2-weighted axial images were also acquired by using a fast spin echo (FSE). The sequence parameters were; TR/TE=4000/14.5 ms, effective TE=58 ms, echo train length (ETL)=8, matrix size=384×384, NEX=8. For both sequences of SPGR and FSE, slices (10-15 of slices) were acquired with a slice thickness of 0.5 mm. The typical field of view (FOV) was 70×70 mm. The total scan time per mouse was about 1 hour.

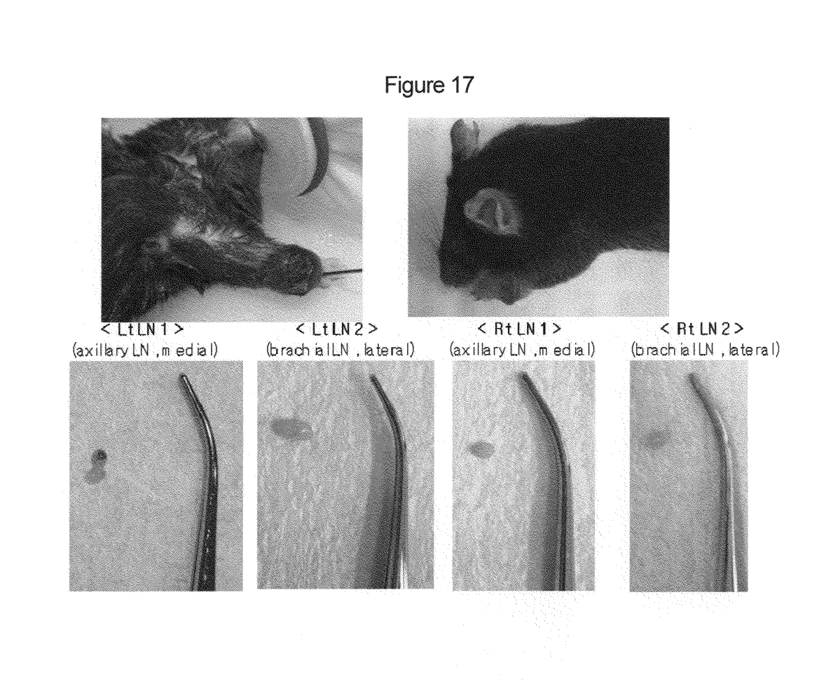

Injected B16F1 cells resulted in the metastases to ipsilateral brachial and axillary lymph nodes. About 14 days after the implantation of B16F1 cells, the size of primary tumor reached about 1 cm in diameter (FIG. 17).

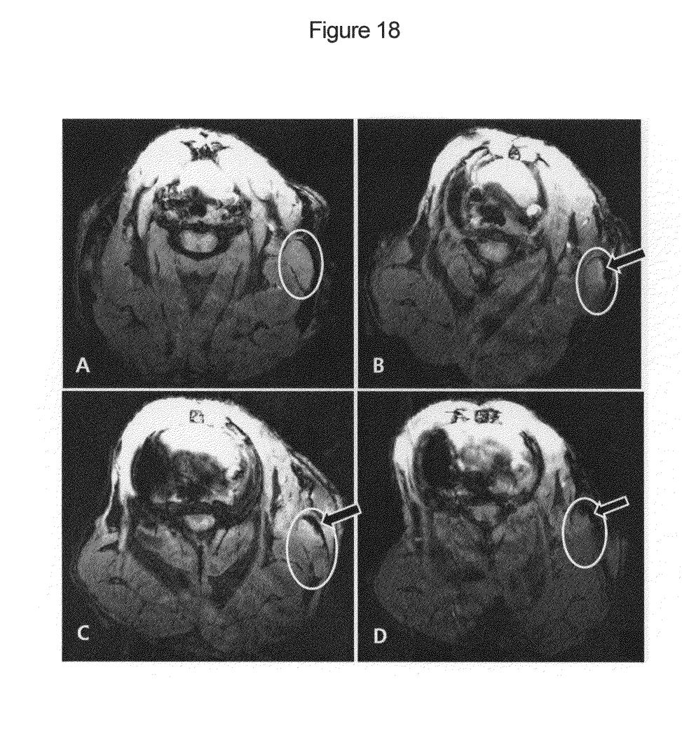

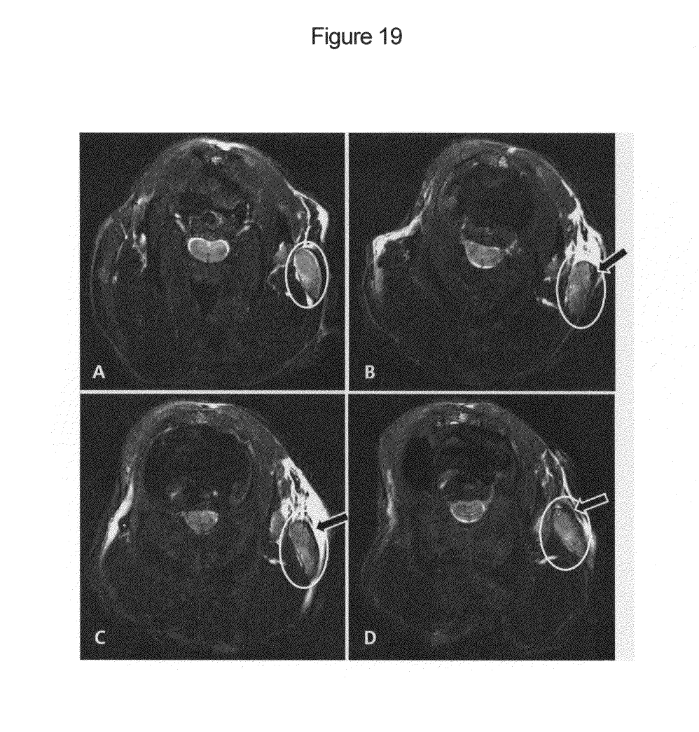

The T1-weighted and T2-weighted MR images of brachial lymph node are presented in Figure FIG. 18 and FIG. 19, respectively. In both imaging protocols, the hypointense regions are clearly suggested that Fe-phytate complex is accumulated in the region of sentinel lymph node. The hypointense signals of sentinel lymph node are shown at 4 hrs to 24 hrs (FIG. 18B-D and FIG. 19B-D) after the administration of Fe-phytate.

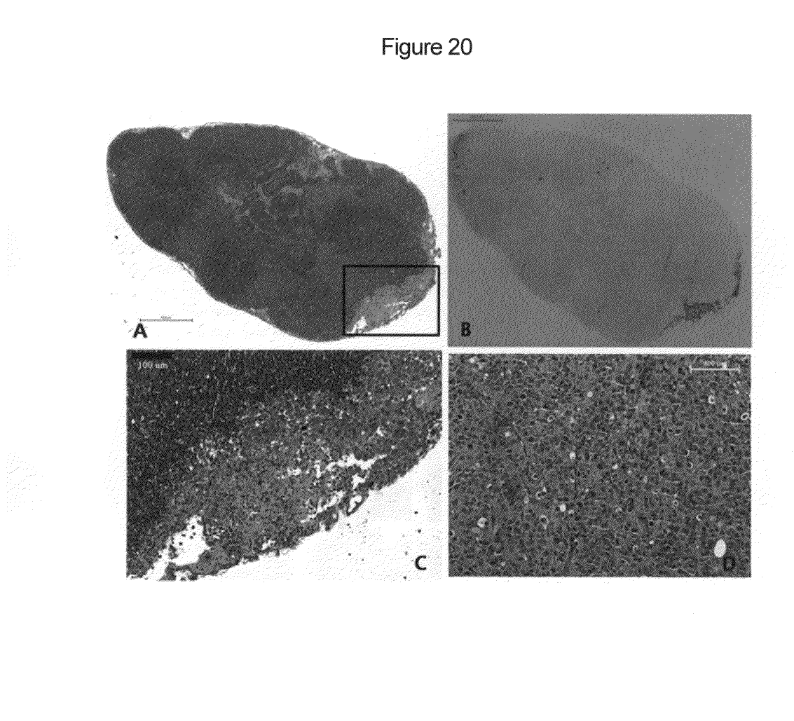

The histopathological results of the left brachial lymph node from the same mouse that was used for acquiring the MR images (FIGS. 18 and 19) are shown in FIG. 20. The morphology of the metastatic site in the lymph node (FIGS. 20 A and B) is in close agreement with that of the primary tumor (FIG. 20D). In FIG. 20B, the regions with the metastasized cancer cells were positively stained by Prussian Blue, which has been used for the staining of Fe³⁺ ions. These data strongly support that the hypointense regions in the MR images (FIG. 18 and FIG. 19) resulted from the accumulation of Fe-phytate complex around the metastatic tumor regions in the sentinel lymph node. Therefore, Fe-phytate complex can detect metastatic lymph nodes by MRI in the mice model of melanoma cancer and can be advantageous over PET and MRI with conventional nanoparticle (SPIO) for the detection of metastatic lymph nodes in clinical application for human patients.

### REFERENCES

Caffrey J J, Hidaka K, Matsuda M, Hirata M, Shears SB. 1999. The human and rat forms of multiple inositol polyphosphate phosphatase: functional homology with a histidine acid phosphatase up-regulated during endochondral ossification. FEBS Lett 442:99-104
Fukuda M, Egawa M, Imao T, Takashima H, Yokoyama K, Namiki M. 2007. Detection of sentinel node micrometastasis by step section and immunohistochemistry in patients with prostate cancer. J Urol 177:1313-7; discussion 7
Graf E, Eaton J W. 1990. Antioxidant functions of phytic acid. Free Radic Biol Med 8:61-9
Grases F, Prieto R M, Simonet B M, March J G. 2000a. Phytate prevents tissue calcifications in female rats. Biofactors 11:171-7
Grases F, Simonet B M, March J G, Prieto R M. 2000b. Inositol hexakisphosphate in urine: the relationship between oral intake and urinary excretion. BJU Int 85:138-42
Hamm B, Staks T, Taupitz M, Maibauer R, Speidel A, et al. 1994. Contrast-enhanced MR imaging of liver and spleen: first experience in humans with a new superparamagnetic iron oxide. J Magn Reson Imaging 4:659-68
Hanakahi L A, Bartlet-Jones M, Chappell C, Pappin D, West S C. 2000. Binding of inositol phosphate to DNA-PK and stimulation of double-strand break repair. Cell 102:721-9
Hino M, Sano M, Sato N, Homma K. 2008. Sentinel lymph node biopsy after neoadjuvant chemotherapy in a patient with operable breast cancer. Surg Today 38:585-91
Hoefs J, Chang K, Wang F, Kanel G, Morgan T, Braunstein P. 1995a. Perfused Kupffer cell mass. Correlation with histology and severity of chronic liver disease. Dig Dis Sci 40:552-60
Hoefs J C, Wang F, Kanel G. 1997. Functional measurement of nonfibrotic hepatic mass in cirrhotic patients. Am J Gastroenterol 92:2054-8
Hoefs J C, Wang F, Kanel G, Braunstein P. 1995b. The liver-spleen scan as a quantitative liver function test: correlation with liver severity at peritoneoscopy. Hepatology 22:1113-21
Huet P M, Chartrand R, Marleau D. 1980. Extrahepatic uptake of 99mTc-phytate: its mechanism and significance in chronic liver disease. Gastroenterology 78:76-80
Ichihara H, Kinoshita F, Hiyoshi K, Beppu T, Fujigasaki K. 2003. [Usefulness of imaging posture using modified oblique view of the axilla (MOVA) for sentinel lymph node scintigraphy in patients with breast cancer]. Nippon Hoshasen Gijutsu Gakkai Zasshi 59:765-70
Ikeda T, Jinno H, Fujii H, Kitajima M. 2004. Recent development of sentinel lymph node biopsy for breast cancer in Japan. Asian J Surg 27:275-8
Kennedy A R. 1995. The evidence for soybean products as cancer preventive agents. J Nutr 125:733 S-43S
Kennedy A R, Manzone H. 1995. Effects of protease inhibitors on levels of proteolytic activity in normal and premalignant cells and tissues. J Cell Biochem Suppl 22:188-94
Kinoshita T. 2007. Sentinel lymph node biopsy is feasible for breast cancer patients after neoadjuvant chemotherapy. Breast Cancer 14:10-5
Koizumi M, Koyama M, Yamashita T, Tada K I, Nishimura S I, et al. 2006. Experience with intradermal injection and intradermal-plus-deep injection in the radioguided sentinel node biopsy of early breast cancer patients. Eur J Surg Oncol 32:738-42
Koizumi M, Nomura E, Yamada Y, Takiguchi T, Ishii M, et al. 2004a. Improved detection of axillary hot nodes in lymphoscintigraphy in breast cancer located in the upper lateral quadrant with additional projection imaging. Ann Nucl Med 18:707-10
Koizumi M, Nomura E, Yamada Y, Takiguchi T, Makita M, et al. 2004b. Radioguided sentinel node detection in breast cancer patients: comparison of 99mTc phytate and 99mTc rhenium colloid efficacy. Nucl Med Commun 25:1031-7
Kosuda S, Kusano S, Kohno N, Ohno Y, Tanabe T, et al. 2003. Feasibility and cost-effectiveness of sentinel lymph node radiolocalization in stage NO head and neck cancer. Arch Otolaryngol Head Neck Surg 129:1105-9
Low R N. 1997. Contrast agents for MR imaging of the liver. J Magn Reson Imaging 7:56-67
Masiero P R, Xavier N L, Spiro B L, Detanico M F, Xavier Mda C, Pinto A L. 2005. Scintigraphic sentinel node detection in breast cancer patients: paired and blinded comparison of 99mTc dextran 500 and 99mTc phytate. Nucl Med Commun 26:1087-91
Modo M M J, BulteAime J. W. M. 2007. Paramagnetic Contrast Agent. Molecular and Cellular MR imaging, CRC Press 1st:37-58
Morota S, Koizumi M, Koyama M, Sugihara T, Tada K I, et al. 2006. Radioactivity thresholds for sentinel node biopsy in breast cancer. Eur J Surg Oncol 32:1101-4
Nakayama H, Shimizu M, Yamada T, Saji H, Sugiura K, Kato H. 2004. [Sentinel node navigation surgery in uterine cancer]. Gan To Kagaku Ryoho 31:2199-202
Niikura H, Okamura C, Akahira J, Takano T, Ito K, et al. 2004a. Sentinel lymph node detection in early cervical cancer with combination 99mTc phytate and patent blue. Gynecol Oncol 94:528-32
Niikura H, Okamura C, Utsunomiya H, Yoshinaga K, Akahira J, et al. 2004b. Sentinel lymph node detection in patients with endometrial cancer. Gynecol Oncol 92:669-74
Niikura H, Yaegashi: N. 2004. [Sentinel lymph node detection in endometrial cancer]. Nippon Rinsho 62 Suppl 10:391-5
Noguchi M. 2001. Sentinel lymph node biopsy in breast cancer: an overview of the Japanese experience. Breast Cancer 8:184-94
Ohno Y, Kohno N; Kanaya T, Nakamura K, Tanabe T, et al. 2005. [Identification of sentinel lymph node in neck-node-negative oral and pharyngeal carcinoma study of patients, it's feasibility, and problems]. Nippon Jibiinkoka Gakkai Kaiho 108:522-7
Ohta K, Takeuchi Y, Tozaki M, Iida T, Ohshita T, et al. 2004. [Efficacy of three-dimensional image fusion of lymphoscintigraphy and MDCT data sets in breast cancer]. Nippon Hoshasen Gijutsu Gakkai Zasshi 60:1424-8
Ohtake E, Asaga T, Inaba M. 2005. Sentinel lymphoscintigraphy in patients with breast cancer undergoing excisional biopsy. Ann Nucl Med 19:671-5
Reddy N R, Sathe S K, Salunkhe D K. 1982. Phytates in legumes and cereals. Adv Food Res 28:1-92
Silva L B, Silva-Filho A L, Traiman P, Triginelli S A, de Lima C F, et al. 2005. Sentinel node detection in cervical cancer with (99m)Tc-phytate. Gynecol Oncol 97:588-95
Stoll G, Wesemeier C, Gold R, Solymosi L, Toyka K V, Bendszus M. 2004. In vivo monitoring of macrophage infiltration in experimental autoimmune neuritis by magnetic resonance imaging. Journal of neuroimmunology 149:142-6
Takashima H, Egawa M, Imao T, Fukuda M, Yokoyama K, Namiki M. 2004. Validity of sentinel lymph node concept for patients with prostate cancer. J Urol 171:2268-71
Takei H, Suemasu K, Kurosumi M, Ninomiya J, Horii Y, et al. 2006. 99mTc-phytate is better than 99mTc-human serum albumin as a radioactive tracer for sentinel lymph node biopsy in breast cancer. Surg Today 36:219-24
Takei H, Suemasu K, Kurosumi M, Uchida K, Igarashi K, et al. 2002. Sentinel lymph node biopsy without axillary dissection after an intraoperative negative histological investigation in 358 invasive breast cancer cases. Breast Cancer 9:344-8
Tavares M G, Sapienza M T, Galeb N A, Jr., Belfort F A, Costa R R, et al. 2001. The use of 99mTc-phytate for sentinel node mapping in melanoma, breast cancer and vulvar cancer: a study of 100 cases. Eur J Nucl Med 28:1597-604
Thompson L U. 1994. Antioxidants and hormone-mediated health benefits of whole grains. Crit. Rev Food Sci Nutr 34:473-97
T J O L, Wei J Y, Tache Y. 1997. Intracisternal TRH and RX 77368 potently activate gastric vagal efferent discharge in rats. Peptides 18:213-9
Torre M, Rodriguez A R, Saura-Calixto F. 1991. Effects of dietary fiber and phytic acid on mineral availability. Crit. Rev Food Sci Nutr 30:1-22
Tozaki M, Uchida K, Yamashita A, Oota K, Takeuchi Y, et al. 2003. [Three-dimensional fusion imaging of lymphoscintigraphy and MDCT for sentinel node biopsy in breast cancer]. Nippon Igaku Hoshasen Gakkai Zasshi 63:412-4
Tsunoda N, Iwata H, Sarumaru S, Mizutani M, Iwase T, Miura S. 2002. Combination of subareolar blue dye and peritumoral RI for sentinel lymph node biopsy. Breast Cancer 9:323-8
Wada N, Sakemura N, Imoto S, Hasebe T, Ochiai A, Moriyama N. 2007. Sentinel node biopsy in primary breast cancer: radioactive detection and metastatic disease. Eur J Surg Oncol 33:691-5
York J D, Odom A R, Murphy R, Ives E B, Wente S R. 1999. A phospholipase C-dependent inositol polyphosphate kinase pathway required for efficient messenger RNA export. Science 285:96-100
Yoshida K, Yamamoto N, Imanaka N, Togawa T, Miyauchi M, Miyazaki M. 2002. Will subareolar injection be a standard technique for sentinel lymph node biopsy, Breast Cancer 9:319-22

All of the references cited herein are incorporated by reference in their entirety.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not to narrow the scope thereof.

1. A magnetic resonance imaging (MRI) contrast agent, comprising a chelating molecule comprising a chelating molecule having at least two phosphate groups coordinated with at least one paramagnetic metal cations.

2. The MRI contrast agent according to claim 1, wherein the chelating molecule is selected from a group consisting of phytate, inositol phosphate and phosphatidylinositol phosphate.

3. The MRI contrast agent according to claim 2, wherein the phytate is d-myo-inositol-1,2,3,4,5,6-hexakisphosphate.

4. The MRI contrast agent according to claim 2, wherein the inositol phosphate is selected from a group consisting of d-myo-inositol-1,2,3,4,5-pentaphosphate, d-myo-inositol-1,3,4,5-tetraphosphate, d-myo-inositol-1,4,5-trisphosphate and d-myo-inositol-1,3,4-trisphosphate.

5. The MRI contrast agent according to claim 2, wherein the phosphatidylinositol phosphate is selected from a group consisting of phosphatidylinositol-3,4,5-trisphosphate and phosphatidylinositol-4,5-bisphosphate.

6. The MRI contrast agent according to claim 1, wherein the paramagnetic metal cation is a transition element.

7. The MRI contrast agent according to claim 6, wherein the transition element is selected from a group consisting of Cr³⁺, Co²⁺, Mn²⁺, Ni²⁺, Fe³⁺, Cu²⁺, and Cu³⁺.

8. The MRI contrast agent according to claim 7, wherein the transition element is selected from a group consisting of Fe²⁺, Fe³⁺, and Mn²⁺.

9. The MRI contrast agent according to claim 1, wherein the paramagnetic metal cation is a lanthanide element.

10. The MRI contrast agent according to claim 9, wherein the lanthanide element is selected from a group consisting of La³⁺, Gd³⁺, Ce³⁺, Tb³⁺, Pr³⁺, Dy³⁺, Nd³⁺, Ho³⁺, Pm³⁺, Er³⁺, Sm³⁺, Tm³⁺, Eu³⁺, Yb³⁺, and Lu³⁺.

11. The MRI contrast agent according to claim 9, wherein the lanthanide element is Gd³⁺.

12. The MRI contrast agent according to claim 10, designed to reduce T1 or T2 relaxation time near a region of interest within a body of a subject.

13. The MRI contrast agent according to 1, wherein the paramagnetic metal cations are used as a radioactive form of paramagnetic metal cations or the paramagnetic metal cations are used with ⁹⁹ Technetium ion as a PET-MRI contrast agent.

14. The MRI contrast agent according to claim 1, wherein the chelating molecule ranges in the pH from 4 to 9.

15. The MRI contrast agent according to claim 14, wherein the chelating molecule ranges in the pH from 6 to 8.

16. The MRI contrast agent according to claim 1, wherein the paramagnetic metal ion is present at a molar ratio of from 0.5 to 3 to the chelating molecule.

17. The MRI contrast agent according to claim 1, wherein a molar ratio of the chelating molecule to the paramagnetic metal ion ranges from 0.5 to 3.

18. The MRI contrast agent according to claim 1, further comprising calcium in an amount corresponding to an equimolar ratio of the MRI contrast agent.

19. A method of visualizing an organ or tissue of a subject in need thereof on a magnetic resonance image, comprising administering the MRI contrast agent according to claim 1 to the subject and imaging the organ or tissue.

20. The method according to claim 19, wherein the MRI contrast agent is administered intravenously when the object is a fatty liver, liver cirrhosis or atherosclerotic plaque.

21. The method according to claim 19, wherein the MRI contrast agent is administered subcutaneously when the object is a metastatic sentinel lymph node of various tumor.

22. The method according to claim 21, wherein the MRI contrast agent is administered subcutaneously when the object is a tumor.

23. The method according to claim 21, wherein the tumor is selected from a group consisting of breast cancer, malignant melanoma, vulvar cancer, squamous cell carcinoma (SCC) of the head and neck, endometrial cancer, cervical cancer, pharyngeal carcinoma, liver cancer, gastric cancer, colorectal cancer, or prostate cancer.

24. The method according to claim 24, wherein the MRI contrast agent is Fe-phytate complex, and the Fe-phytate complex is attached with anticancer drug.

25. The method according to claim 24, wherein the anticancer drug is doxorubicin.

26. A method for detecting macrophage activity in an organ or tissue of interest on a magnetic resonance image, comprising the MRI contrast agent according to claim 1 to a subject in need thereof and imaging the organ or tissue, whereby infection or inflammation of the organ or tissue can be diagnosed.

27. A method for non-invasively monitoring macrophage infiltration into sites of inflammation on a magnetic resonance image, comprising administering the MRI contrast agent according to claim 1 to a subject in need thereof, and imaging the sites.

28. A method for visualizing presence of transplanted cells in vivo on a magnetic resonance image, comprising:
inserting the MRI contrast agent according to claim 1 into the cells;
transplanting the cells to a site in a subject; and
imaging the site using magnetic resonance imaging.

29. A method for co-registering contrast agent for PET-MRI scanner images, comprising:
inserting the PET-MRI contrast agent according to claim 13 into the cells;
transplanting the cells to a site in a subject; and
imaging the site using PET-MRI scanner.
